SEQUENCE LISTING (66 sequences)

SEQ ID NO: 1 - DNA, 13 bp, synthetic construct
  source 1..13: mol_type = other DNA
cagccgagccaat

SEQ ID NO: 2 - DNA, 18 bp, synthetic construct
  source 1..18: mol_type = other DNA
caccagccgagccaattc

SEQ ID NO: 3 - DNA, 15 bp, synthetic construct
  source 1..15: mol_type = other DNA
ctgagcaaattcatg

SEQ ID NO: 4 - DNA, 20 bp, synthetic construct
  source 1..20: mol_type = other DNA
cagctgagcaaattcatgga

SEQ ID NO: 5 - DNA, 14 bp, synthetic construct
  source 1..14: mol_type = other DNA
ctgagcccattcat

SEQ ID NO: 6 - DNA, 19 bp, synthetic construct
  source 1..19: mol_type = other DNA
cagctgagcccattcatgg

SEQ ID NO: 7 - DNA, 18 bp, synthetic construct
  source 1..18: mol_type = other DNA
aattcatggtccagaaca

SEQ ID NO: 8 - DNA, 23 bp, synthetic construct
  source 1..23: mol_type = other DNA
gccaattcatggtccagaacaac

SEQ ID NO: 9 - DNA, 17 bp, synthetic construct
  source 1..17: mol_type = other DNA
ttcatgtaccagaacaa

SEQ ID NO: 10 - DNA, 23 bp, synthetic construct
  source 1..23: mol_type = other DNA
caattcatgtaccagaacaaccc

SEQ ID NO: 11 - DNA, 14 bp, synthetic construct
  source 1..14: mol_type = other DNA
cgcttgtcggtcaa

SEQ ID NO: 12 - DNA, 19 bp, synthetic construct
  source 1..19: mol_type = other DNA
cggcgcttgtcggtcaacc

SEQ ID NO: 13 - DNA, 14 bp, synthetic construct
  source 1..14: mol_type = other DNA
cttgagggtcaacc

SEQ ID NO: 14 - DNA, 19 bp, synthetic construct
  source 1..19: mol_type = other DNA
gcgcttgagggtcaacccc

SEQ ID NO: 15 - DNA, 13 bp, synthetic construct
  source 1..13: mol_type = other DNA
cttgcgggtcaac

SEQ ID NO: 16 - DNA, 18 bp, synthetic construct
  source 1..18: mol_type = other DNA
gcgcttgcgggtcaaccc

SEQ ID NO: 17 - DNA, 15 bp, synthetic construct
  source 1..15: mol_type = other DNA
cttgtaggtcaaccc

SEQ ID NO: 18 - DNA, 20 bp, synthetic construct
  source 1..20: mol_type = other DNA
gcgcttgtaggtcaaccccg

SEQ ID NO: 19 - DNA, 13 bp, synthetic construct
  source 1..13: mol_type = other DNA
ccgactgttggcg

SEQ ID NO: 20 - DNA, 18 bp, synthetic construct
  source 1..18: mol_type = other DNA
gcgccgactgttggcgct

SEQ ID NO: 21 - DNA, 13 bp, synthetic construct
  source 1..13: mol_type = other DNA
cgactgtgggcgc

SEQ ID NO: 22 - DNA, 18 bp, synthetic construct
  source 1..18: mol_type = other DNA
cgccgactgtgggcgctg

SEQ ID NO: 23 - DNA, 14 bp, synthetic construct
  source 1..14: mol_type = other DNA
actgtcggcgccgg

SEQ ID NO: 24 - DNA, 19 bp, synthetic construct
  source 1..19: mol_type = other DNA
ccgactgtcggcgccgggg

SEQ ID NO: 25 - DNA, 13 bp, synthetic construct
  source 1..13: mol_type = other DNA
  modified_base 2: mod_base = OTHER; note = D-Locked Nucleic Acid
  modified_base 4..5: mod_base = OTHER; note = D-Locked Nucleic Acid
  modified_base 7: mod_base = OTHER; note = D-Locked Nucleic Acid
  modified_base 9: mod_base = OTHER; note = D-Locked Nucleic Acid
cagccgagccaat

SEQ ID NO: 26 - DNA, 18 bp, synthetic construct
  source 1..18: mol_type = other DNA
  modified_base 5: mod_base = OTHER; note = D-Locked Nucleic Acid
  modified_base 7..8: mod_base = OTHER; note = D-Locked Nucleic Acid
  modified_base 10: mod_base = OTHER; note = D-Locked Nucleic Acid
  modified_base 12: mod_base = OTHER; note = D-Locked Nucleic Acid
caccagccgagccaattc

SEQ ID NO: 27 - DNA, 15 bp, synthetic construct
  source 1..15: mol_type = other DNA
  modified_base 2: mod_base = OTHER; note = D-Locked Nucleic Acid
  modified_base 4: mod_base = OTHER; note = D-Locked Nucleic Acid
  modified_base 6..7: mod_base = OTHER; note = D-Locked Nucleic Acid
  modified_base 9: mod_base = OTHER; note = D-Locked Nucleic Acid
  modified_base 11: mod_base = OTHER; note = D-Locked Nucleic Acid
ctgagcaaattcatg

SEQ ID NO: 28 - DNA, 20 bp, synthetic construct
  source 1..20: mol_type = other DNA
  modified_base 5: mod_base = OTHER; note = D-Locked Nucleic Acid
  modified_base 7: mod_base = OTHER; note = D-Locked Nucleic Acid
  modified_base 9..10: mod_base = OTHER; note = D-Locked Nucleic Acid
  modified_base 12: mod_base = OTHER; note = D-Locked Nucleic Acid
  modified_base 14: mod_base = OTHER; note = D-Locked Nucleic Acid
cagctgagcaaattcatgga

SEQ ID NO: 29 - DNA, 14 bp, synthetic construct
  source 1..14: mol_type = other DNA
  modified_base 3: mod_base = OTHER; note = D-Locked Nucleic Acid
  modified_base 5: mod_base = OTHER; note = D-Locked Nucleic Acid
  modified_base 7..8: mod_base = OTHER; note = D-Locked Nucleic Acid
  modified_base 10: mod_base = OTHER; note = D-Locked Nucleic Acid
  modified_base 12: mod_base = OTHER; note = D-Locked Nucleic Acid
ctgagcccattcat

SEQ ID NO: 30 - DNA, 19 bp, synthetic construct
  source 1..19: mol_type = other DNA
  modified_base 6: mod_base = OTHER; note = D-Locked Nucleic Acid
  modified_base 8: mod_base = OTHER; note = D-Locked Nucleic Acid
  modified_base 10..11: mod_base = OTHER; note = D-Locked Nucleic Acid
  modified_base 13: mod_base = OTHER; note = D-Locked Nucleic Acid
  modified_base 15: mod_base = OTHER; note = D-Locked Nucleic Acid
cagctgagcccattcatgg

SEQ ID NO: 31 - DNA, 18 bp, synthetic construct
  source 1..18: mol_type = other DNA
  modified_base 7: mod_base = OTHER; note = D-Locked Nucleic Acid
  modified_base 9..10: mod_base = OTHER; note = D-Locked Nucleic Acid
  modified_base 12: mod_base = OTHER; note = D-Locked Nucleic Acid
aattcatggtccagaaca

SEQ ID NO: 32 - DNA, 18 bp, synthetic construct
  source 1..18: mol_type = other DNA
  modified_base 5: mod_base = OTHER; note = D-Locked Nucleic Acid
  modified_base 7: mod_base = OTHER; note = D-Locked Nucleic Acid
  modified_base 9..10: mod_base = OTHER; note = D-Locked Nucleic Acid
  modified_base 12: mod_base = OTHER; note = D-Locked Nucleic Acid
aattcatggtccagaaca

SEQ ID NO: 33 - DNA, 18 bp, synthetic construct
  source 1..18: mol_type = other DNA
  modified_base 5: mod_base = OTHER; note = D-Locked Nucleic Acid
  modified_base 7: mod_base = OTHER; note = D-Locked Nucleic Acid
  modified_base 9..10: mod_base = OTHER; note = D-Locked Nucleic Acid
  modified_base 12: mod_base = OTHER; note = D-Locked Nucleic Acid
  modified_base 14: mod_base = OTHER; note = D-Locked Nucleic Acid
aattcatggtccagaaca

SEQ ID NO: 34 - DNA, 23 bp, synthetic construct
  source 1..23: mol_type = other DNA
  modified_base 10: mod_base = OTHER; note = D-Locked Nucleic Acid
  modified_base 12..13: mod_base = OTHER; note = D-Locked Nucleic Acid
  modified_base 15: mod_base = OTHER; note = D-Locked Nucleic Acid
gccaattcatggtccagaacaac

SEQ ID NO: 35 - DNA, 23 bp, synthetic construct
  source 1..23: mol_type = other DNA
  modified_base 8: mod_base = OTHER; note = D-Locked Nucleic Acid
  modified_base 10: mod_base = OTHER; note = D-Locked Nucleic Acid
  modified_base 12..13: mod_base = OTHER; note = D-Locked Nucleic Acid
  modified_base 15: mod_base = OTHER; note = D-Locked Nucleic Acid
gccaattcatggtccagaacaac

SEQ ID NO: 36 - DNA, 23 bp, synthetic construct
  source 1..23: mol_type = other DNA
  modified_base 8: mod_base = OTHER; note = D-Locked Nucleic Acid
  modified_base 10: mod_base = OTHER; note = D-Locked Nucleic Acid
  modified_base 12..13: mod_base = OTHER; note = D-Locked Nucleic Acid
  modified_base 15: mod_base = OTHER; note = D-Locked Nucleic Acid
  modified_base 17: mod_base = OTHER; note = D-Locked Nucleic Acid
gccaattcatggtccagaacaac

SEQ ID NO: 37 - DNA, 17 bp, synthetic construct
  source 1..17: mol_type = other DNA
  modified_base 2: mod_base = OTHER; note = D-Locked Nucleic Acid
  modified_base 4: mod_base = OTHER; note = D-Locked Nucleic Acid
  modified_base 6..7: mod_base = OTHER; note = D-Locked Nucleic Acid
  modified_base 9: mod_base = OTHER; note = D-Locked Nucleic Acid
  modified_base 11: mod_base = OTHER; note = D-Locked Nucleic Acid
ttcatgtaccagaacaa

SEQ ID NO: 38 - DNA, 23 bp, synthetic construct
  source 1..23: mol_type = other DNA
  modified_base 5: mod_base = OTHER; note = D-Locked Nucleic Acid
  modified_base 7: mod_base = OTHER; note = D-Locked Nucleic Acid
  modified_base 9..10: mod_base = OTHER; note = D-Locked Nucleic Acid
  modified_base 12: mod_base = OTHER; note = D-Locked Nucleic Acid
  modified_base 14: mod_base = OTHER; note = D-Locked Nucleic Acid
caattcatgtaccagaacaaccc

SEQ ID NO: 39 - DNA, 14 bp, synthetic construct
  source 1..14: mol_type = other DNA
  modified_base 5: mod_base = OTHER; note = D-Locked Nucleic Acid
  modified_base 7..8: mod_base = OTHER; note = D-Locked Nucleic Acid
  modified_base 10: mod_base = OTHER; note = D-Locked Nucleic Acid
cgcttgtcggtcaa

SEQ ID NO: 40 - DNA, 14 bp, synthetic construct
  source 1..14: mol_type = other DNA
  modified_base 3: mod_base = OTHER; note = D-Locked Nucleic Acid
  modified_base 5: mod_base = OTHER; note = D-Locked Nucleic Acid
  modified_base 7..8: mod_base = OTHER; note = D-Locked Nucleic Acid
  modified_base 10: mod_base = OTHER; note = D-Locked Nucleic Acid
cgcttgtcggtcaa

SEQ ID NO: 41 - DNA, 14 bp, synthetic construct
  source 1..14: mol_type = other DNA
  modified_base 3: mod_base = OTHER; note = D-Locked Nucleic Acid
  modified_base 5: mod_base = OTHER; note = D-Locked Nucleic Acid
  modified_base 7..8: mod_base = OTHER; note = D-Locked Nucleic Acid
  modified_base 10: mod_base = OTHER; note = D-Locked Nucleic Acid
  modified_base 12: mod_base = OTHER; note = D-Locked Nucleic Acid
cgcttgtcggtcaa

SEQ ID NO: 42 - DNA, 19 bp, synthetic construct
  source 1..19: mol_type = other DNA
  modified_base 8: mod_base = OTHER; note = D-Locked Nucleic Acid
  modified_base 10..11: mod_base = OTHER; note = D-Locked Nucleic Acid
  modified_base 13: mod_base = OTHER; note = D-Locked Nucleic Acid
cggcgcttgtcggtcaacc

SEQ ID NO: 43 - DNA, 19 bp, synthetic construct
  source 1..19: mol_type = other DNA
  modified_base 6: mod_base = OTHER; note = D-Locked Nucleic Acid
  modified_base 8: mod_base = OTHER; note = D-Locked Nucleic Acid
  modified_base 10..11: mod_base = OTHER; note = D-Locked Nucleic Acid
  modified_base 13: mod_base = OTHER; note = D-Locked Nucleic Acid
cggcgcttgtcggtcaacc

SEQ ID NO: 44 - DNA, 19 bp, synthetic construct
  source 1..19: mol_type = other DNA
  modified_base 6: mod_base = OTHER; note = D-Locked Nucleic Acid
  modified_base 8: mod_base = OTHER; note = D-Locked Nucleic Acid
  modified_base 10..11: mod_base = OTHER; note = D-Locked Nucleic Acid
  modified_base 13: mod_base = OTHER; note = D-Locked Nucleic Acid
  modified_base 15: mod_base = OTHER; note = D-Locked Nucleic Acid
cggcgcttgtcggtcaacc

SEQ ID NO: 45 - DNA, 14 bp, synthetic construct
  source 1..14: mol_type = other DNA
  modified_base 2: mod_base = OTHER; note = D-Locked Nucleic Acid
  modified_base 4..5: mod_base = OTHER; note = D-Locked Nucleic Acid
  modified_base 7: mod_base = OTHER; note = D-Locked Nucleic Acid
  modified_base 9: mod_base = OTHER; note = D-Locked Nucleic Acid
  modified_base 11: mod_base = OTHER; note = D-Locked Nucleic Acid
cttgagggtcaacc

SEQ ID NO: 46 - DNA, 19 bp, synthetic construct
  source 1..19: mol_type = other DNA
  modified_base 5: mod_base = OTHER; note = D-Locked Nucleic Acid
  modified_base 7..8: mod_base = OTHER; note = D-Locked Nucleic Acid
  modified_base 10: mod_base = OTHER; note = D-Locked Nucleic Acid
  modified_base 12: mod_base = OTHER; note = D-Locked Nucleic Acid
  modified_base 14: mod_base = OTHER; note = D-Locked Nucleic Acid
gcgcttgagggtcaacccc

SEQ ID NO: 47 - DNA, 13 bp, synthetic construct
  source 1..13: mol_type = other DNA
  modified_base 2: mod_base = OTHER; note = D-Locked Nucleic Acid
  modified_base 4..5: mod_base = OTHER; note = D-Locked Nucleic Acid
  modified_base 7: mod_base = OTHER; note = D-Locked Nucleic Acid
  modified_base 9: mod_base = OTHER; note = D-Locked Nucleic Acid
cttgcgggtcaac

SEQ ID NO: 48 - DNA, 18 bp, synthetic construct
  source 1..18: mol_type = other DNA
  modified_base 5: mod_base = OTHER; note = D-Locked Nucleic Acid
  modified_base 7..8: mod_base = OTHER; note = D-Locked Nucleic Acid
  modified_base 10: mod_base = OTHER; note = D-Locked Nucleic Acid
  modified_base 12: mod_base = OTHER; note = D-Locked Nucleic Acid
gcgcttgcgggtcaaccc

SEQ ID NO: 49 - DNA, 15 bp, synthetic construct
  source 1..15: mol_type = other DNA
  modified_base 2: mod_base = OTHER; note = D-Locked Nucleic Acid
  modified_base 4..5: mod_base = OTHER; note = D-Locked Nucleic Acid
  modified_base 7: mod_base = OTHER; note = D-Locked Nucleic Acid
cttgtaggtcaaccc

SEQ ID NO: 50 - DNA, 20 bp, synthetic construct
  source 1..20: mol_type = other DNA
  modified_base 5: mod_base = OTHER; note = D-Locked Nucleic Acid
  modified_base 7..8: mod_base = OTHER; note = D-Locked Nucleic Acid
  modified_base 10: mod_base = OTHER; note = D-Locked Nucleic Acid
gcgcttgtaggtcaaccccg

SEQ ID NO: 51 - DNA, 13 bp, synthetic construct
  source 1..13: mol_type = other DNA
  modified_base 4: mod_base = OTHER; note = D-Locked Nucleic Acid
  modified_base 6: mod_base = OTHER; note = D-Locked Nucleic Acid
  modified_base 8..9: mod_base = OTHER; note = D-Locked Nucleic Acid
  modified_base 11: mod_base = OTHER; note = D-Locked Nucleic Acid
ccgactgttggcg

SEQ ID NO: 52 - DNA, 18 bp, synthetic construct
  source 1..18: mol_type = other DNA
  modified_base 7: mod_base = OTHER; note = D-Locked Nucleic Acid
  modified_base 9: mod_base = OTHER; note = D-Locked Nucleic Acid
  modified_base 11..12: mod_base = OTHER; note = D-Locked Nucleic Acid
  modified_base 14: mod_base = OTHER; note = D-Locked Nucleic Acid
gcgccgactgttggcgct

SEQ ID NO: 53 - DNA, 13 bp, synthetic construct
  source 1..13: mol_type = other DNA
  modified_base 5: mod_base = OTHER; note = D-Locked Nucleic Acid
  modified_base 7..8: mod_base = OTHER; note = D-Locked Nucleic Acid
cgactgtgggcgc

SEQ ID NO: 54 - DNA, 13 bp, synthetic construct
  source 1..13: mol_type = other DNA
  modified_base 5: mod_base = OTHER; note = D-Locked Nucleic Acid
  modified_base 7..8: mod_base = OTHER; note = D-Locked Nucleic Acid
  modified_base 10: mod_base = OTHER; note = D-Locked Nucleic Acid
cgactgtgggcgc

SEQ ID NO: 55 - DNA, 13 bp, synthetic construct
  source 1..13: mol_type = other DNA
  modified_base 3: mod_base = OTHER; note = D-Locked Nucleic Acid
  modified_base 5: mod_base = OTHER; note = D-Locked Nucleic Acid
  modified_base 7..8: mod_base = OTHER; note = D-Locked Nucleic Acid
  modified_base 10: mod_base = OTHER; note = D-Locked Nucleic Acid
cgactgtgggcgc

SEQ ID NO: 56 - DNA, 13 bp, synthetic construct
  source 1..13: mol_type = other DNA
  modified_base 4..5: mod_base = OTHER; note = D-Locked Nucleic Acid
  modified_base 7..8: mod_base = OTHER; note = D-Locked Nucleic Acid
  modified_base 10..11: mod_base = OTHER; note = D-Locked Nucleic Acid
cgactgtgggcgc

SEQ ID NO: 57 - DNA, 16 bp, synthetic construct
  source 1..16: mol_type = other DNA
  modified_base 8: mod_base = OTHER; note = D-Locked Nucleic Acid
  modified_base 10..11: mod_base = OTHER; note = D-Locked Nucleic Acid
cgccgactgtgggcgc

SEQ ID NO: 58 - DNA, 16 bp, synthetic construct
  source 1..16: mol_type = other DNA
  modified_base 8: mod_base = OTHER; note = D-Locked Nucleic Acid
  modified_base 10..11: mod_base = OTHER; note = D-Locked Nucleic Acid
  modified_base 13: mod_base = OTHER; note = D-Locked Nucleic Acid
cgccgactgtgggcgc

SEQ ID NO: 59 - DNA, 18 bp, synthetic construct
  source 1..18: mol_type = other DNA
  modified_base 6: mod_base = OTHER; note = D-Locked Nucleic Acid
  modified_base 8: mod_base = OTHER; note = D-Locked Nucleic Acid
  modified_base 10..11: mod_base = OTHER; note = D-Locked Nucleic Acid
  modified_base 13: mod_base = OTHER; note = D-Locked Nucleic Acid
cgccgactgtgggcgctg

SEQ ID NO: 60 - DNA, 17 bp, synthetic construct
  source 1..17: mol_type = other DNA
  modified_base 6..7: mod_base = OTHER; note = D-Locked Nucleic Acid
  modified_base 9..10: mod_base = OTHER; note = D-Locked Nucleic Acid
  modified_base 12..13: mod_base = OTHER; note = D-Locked Nucleic Acid
cgcgactgtgggcgctg

SEQ ID NO: 61 - DNA, 14 bp, synthetic construct
  source 1..14: mol_type = other DNA
  modified_base 9: mod_base = OTHER; note = D-Locked Nucleic Acid
  modified_base 11..12: mod_base = OTHER; note = D-Locked Nucleic Acid
  modified_base 14: mod_base = OTHER; note = D-Locked Nucleic Acid
actgtcggcgccgg

SEQ ID NO: 62 - DNA, 19 bp, synthetic construct
  source 1..19: mol_type = other DNA
  modified_base 12: mod_base = OTHER; note = D-Locked Nucleic Acid
  modified_base 14..15: mod_base = OTHER; note = D-Locked Nucleic Acid
  modified_base 17: mod_base = OTHER; note = D-Locked Nucleic Acid
ccgactgtcggcgccgggg

SEQ ID NO: 63 - DNA, 20 bp, synthetic construct
  source 1..20: mol_type = other DNA
aggcgatcacaccgcagacg

SEQ ID NO: 64 - DNA, 20 bp, synthetic construct
  source 1..20: mol_type = other DNA
ccgacagcgagccgatcaga

SEQ ID NO: 65 - DNA, 15 bp, synthetic construct
  source 1..15: mol_type = other DNA
catccggccggtggt

SEQ ID NO: 66 - DNA, 18 bp, synthetic construct
  source 1..18: mol_type = other DNA
ggcacgctcacgtgacag

(19) United States
(12) Patent Application Publication
(10) Pub. No.: US 2026/0009086 A1
(43) Pub. Date: Jan. 8, 2026

(54) NUCLEIC ACID PROBE COMBINATION AND METHOD FOR DETECTING DRUG RESISTANCE GENE MUTATION IN MYCOBACTERIUM TUBERCULOSIS

(71) Applicant: Delta Electronics (Shanghai) Co., Ltd., Shanghai (CN)

(72) Inventors: Quan-Ke LONG, Shanghai (CN); Xiao-Zhi ZHAO, Shanghai (CN); Wei-Shi ZHANG, Shanghai (CN); Jiao-Jiao LI, Shanghai (CN)

(21) Appl. No.: 19/257,482

(22) Filed: Jul. 2, 2025

(30) Foreign Application Priority Data
Jul. 2, 2024 (CN) 202410884025.6

(51) Int. Cl. C12Q 1/689; C12Q 1/686

(52) U.S. Cl. CPC C12Q 1/689; C12Q 1/686; C12Q 2600/156

(57) ABSTRACT

A nucleic acid probe combination for detecting rifampicin resistance gene mutations in Mycobacterium tuberculosis, including: a plurality of probes configured for nucleic acid amplification reactions, wherein each of the plurality of probes is completely complementary to one of mutant sequence segments in the drug resistance-determining region of rpoB gene, and at least one of the plurality of probes has a locked nucleic acid (LNA) modification.

### CROSS-REFERENCE TO RELATED APPLICATION

This application claims priority to China Application Serial Number 202410884025.6, filed Jul. 2, 2024, which is herein incorporated by reference in its entirety.

The Sequence Listing associated with this application is filed in electronic format via EFS-Web and is hereby incorporated by reference into the specification in its entirety. The name of the text file containing the Sequence Listing is NP-36466-US_202509_SEQ_LIST.xml. The size of the text file is 139,992 bytes, and the text file was created on Sep. 8, 2025.

### BACKGROUND

### Field of Invention

The present disclosure relates to a nucleic acid probe combination and related detection methods for detecting gene mutations associated with rifampicin resistance of Mycobacterium tuberculosis.

### Description of Related Art

Tuberculosis (TB) is a chronic infectious disease caused by Mycobacterium tuberculosis (MTB) and is the second leading cause of death from infectious diseases after COVID-19. On Oct. 27, 2022, the World Health Organization (WHO) released the latest “Global Tuberculosis Report 2022,” which showed that there were 10.6 million new TB cases globally in 2021, with an incidence rate of 134 per 100,000 people. Currently, the drugs used for TB treatment include rifampicin, isoniazid, ethambutol, pyrazinamide, and streptomycin, etc., but drug resistance is a serious issue. According to the report, the burden of drug-resistant tuberculosis (DR-TB) increased during 2020-2021. In 2021, there were 450,000 new cases of rifampicin-resistant tuberculosis (RR-TB). The types of drugs available for TB treatment are limited, and there is cross-resistance between first-line and second-line TB drugs. Additionally, almost no new treatment drugs introduced in recent years, leading to poor efficacy and unfavorable prognosis. TB brings immense mental and financial pressure to patients and their families, while also causing a significant economic burden to the country.

Rifampicin, as one of the frontline anti-tuberculosis drugs, targets the β subunit of bacterial RNA polymerase. Rifampicin binds to the β subunit of RNA polymerase, hindering mRNA synthesis, inhibiting the transcription process, and blocking bacterial protein synthesis, thereby achieving an antibacterial effect.

In clinical practice, the traditional method for drug susceptibility testing of drug-resistant tuberculosis is still based on culture-based drug sensitivity testing. This method can simultaneously detect the resistance levels to first-line and second-line drugs, but the results may be easily affected by factors such as drug concentration, the inoculum size of Mycobacterium tuberculosis (MTB) and the viability of MTB, leading to inaccurate results. Moreover, the culture-based methods are time-consuming, taking about 1 month on average. On the basis of traditional culture methods, a rapid liquid culture system for Mycobacteria has been developed. This system shortens the conventional culture time from an average of 1 month to 8 to 10 days. However, this system requires specialized equipment, and the imported products for this system are expensive. In addition, the minimum inhibitory concentration (MIC) testing method for Mycobacterium tuberculosis can shorten the testing time to 1 to 2 weeks compared to traditional culture methods. However, in practical use of this method, the MIC of some drugs may be difficult to interpret.

In contrast, molecular diagnostic-based methods for detecting drug resistance can shorten the detection time to within one day, greatly improving detection speed and efficiency. WHO also recommends rapid molecular diagnostic testing to achieve earlier and more accurate diagnosis of TB and drug-resistant TB, and considers the accessibility of such diagnostic testing a crucial aspect of strengthening TB laboratory efforts in the “End TB” Strategy.

However, the use of such rapid testing technology remains very limited. Of the 6.4 million new TB cases diagnosed in 2021, only 38% used WHO-recommended rapid molecular testing, slightly higher than 33% in 2020 and 28% in 2019.

Currently, the main players in the market for molecular diagnostic methods for DR-TB detection include Cepheid, Zhishan Biological Technology, Yaneng Bioscience, Daan Gene, and InnowaveDx. Among them, Cepheid's detection principle involves designing five differently colored fluorescent probes targeting the wild-type templates of rpoB gene segments. These probes can only hybridize with the wild-type rpoB gene sequence segments, and any mutation in the rpoB gene sequence segments will cause a delay or loss of the fluorescent signal. The presence of drug resistance is determined based on the difference in ΔCq values. This method requires high expertise in probe design and ΔCq value judgment algorithms, as the PCR reaction targeting the wild-type template cannot distinguish between different types of mutations. Additionally, the product is costly and may not be affordable in economically underdeveloped regions.

Therefore, in the field of drug resistance testing for Mycobacterium tuberculosis, there is an urgent need to develop testing reagents and methods that are easy to operate, cost-effective, highly efficient, and possess high sensitivity and accuracy to meet clinical demands.

### SUMMARY

In view of the aforementioned issues, the present disclosure provides a nucleic acid probe combination and detection method designed for mutant templates, aiming to enhance the sensitivity of drug resistance detection for tuberculosis.

Some embodiments of the present disclosure provide a nucleic acid probe combination for detecting rifampicin resistance gene mutations in Mycobacterium tuberculosis, including: a plurality of probes for nucleic acid amplification reactions, wherein each of the plurality of probes is completely complementary to one of mutant sequence segments in the drug resistance-determining region of rpoB gene, and at least one of the plurality of probes has a locked nucleic acid (LNA) modification.

In some embodiments, each of the plurality of probes is configured to detect a mutation in one of codon 511, codon 513, codon 516, codon 526, codon 531, or codon 533 of rpoB gene.

In some embodiments, each of the plurality of probes corresponds to one of L511P mutation, Q513K mutation, Q513P mutation, D516V mutation, D516Y mutation, H526D mutation, H526L mutation, H526R mutation, H526Y mutation, S531L mutation, S531W mutation, and L533P mutation.

In some embodiments, the LNA modification includes 3 to 8 LNA-modified bases, and the LNA-modified bases are located on both sides of a mutated base. In some embodiments, the LNA modification includes 4 to 6 LNA-modified bases.

In some embodiments, the LNA modification includes 3 LNA-modified nucleotides within the region of two bases on both sides of a mutated base. In some embodiments, the LNA modification is within the region of two bases on both sides of a mutated base and includes two consecutive LNA-modified nucleotides and another LNA-modified nucleotide spaced apart.

In some embodiments, another probe of the plurality of probes has an MGB modification or is a Taqman probe.

In some embodiments, each of the plurality of probes includes: a fluorescent group at the 5′ end; and a quencher group at the 3′ end. The fluorescent group is FAM, Hex, TET, VIC, Cy5, or Rox, and the quencher group is BHQ1, BHQ2, or BHQ3.

In some embodiments, the plurality of probes include: a first probe including the sequence of SEQ ID NO: 1 or 2; a second probe including the sequence of SEQ ID NO: 3 or 4; a third probe including the sequence of SEQ ID NO: 5 or 6; a fourth probe including the sequence of SEQ ID NO: 7 or 8; a fifth probe including the sequence of SEQ ID NO: 9 or 10; a sixth probe including the sequence of SEQ ID NO: 11 or 12; a seventh probe including the sequence of SEQ ID NO: 13 or 14; an eighth probe including the sequence of SEQ ID NO: 15 or 16; a ninth probe including the sequence of SEQ ID NO: 17 or 18; a tenth probe including the sequence of SEQ ID NO: 19 or 20; an eleventh probe including the sequence of SEQ ID NO: 21 or 22; or a twelfth probe including the sequence of SEQ ID NO: 23 or 24.

In some embodiments, each of the first probe, second probe, third probe, fourth probe, fifth probe, sixth probe, seventh probe, eighth probe, ninth probe, tenth probe, eleventh probe, and twelfth probe has a plurality of LNA-modified bases.

In some embodiments, the first probe has the sequence of SEQ ID NO: 1 or 2 and has multiple LNA-modified bases; the second probe has the sequence of SEQ ID NO: 3 or 4 and has multiple LNA-modified bases; the third probe has the sequence of SEQ ID NO: 5 or 6 and has multiple LNA-modified bases; the fourth probe has the sequence of SEQ ID NO: 7 or 8 and has multiple LNA-modified bases; the fifth probe has the sequence of SEQ ID NO: 9 or 10 and has multiple LNA-modified bases; the sixth probe has the sequence of SEQ ID NO: 11 or 12 and has multiple LNA-modified bases; the seventh probe has the sequence of SEQ ID NO: 13 or 14 and has multiple LNA-modified bases; the eighth probe has the sequence of SEQ ID NO: 15 or 16 and has multiple LNA-modified bases; the ninth probe has the sequence of SEQ ID NO: 17 or 18 and has multiple LNA-modified bases; the tenth probe has the sequence of SEQ ID NO: 19 or 20 and has multiple LNA-modified bases; the eleventh probe has the sequence of SEQ ID NO: 21 or 22 and has multiple LNA-modified bases; or the twelfth probe has the sequence of SEQ ID NO: 23 or 24 and has multiple LNA-modified bases.

In some embodiments, the first probe has the sequence of SEQ ID NO: 25 or 26; the second probe has the sequence of SEQ ID NO: 27 or 28; the third probe has the sequence of SEQ ID NO: 29 or 30; the fourth probe has the sequence of one of SEQ ID NO: 31 to 36; the fifth probe has the sequence of SEQ ID NO: 37 or 38; the sixth probe has the sequence of one of SEQ ID NO: 39 to 44; the seventh probe has the sequence of SEQ ID NO: 45 or 46; the eighth probe has the sequence of SEQ ID NO: 47 or 48; the ninth probe has the sequence of SEQ ID NO: 49 or 50; the tenth probe has the sequence of SEQ ID NO: 51 or 52; the eleventh probe has the sequence of one of SEQ ID NO: 53 to 60; and the twelfth probe has the sequence of SEQ ID NO: 61 or 62.

Other embodiments of the present disclosure provide a method for detecting rifampicin resistance gene mutations in Mycobacterium tuberculosis, including: obtaining a DNA sample to be tested; performing a nucleic acid amplification reaction, including: performing the nucleic acid amplification reaction using a nucleic acid probe combination, wherein the nucleic acid probe combination includes a plurality of probes, wherein each of the plurality of probes is completely complementary to one of mutant sequence segments in the drug resistance-determining region of rpoB gene, and at least one of the plurality of probes has LNA modification.

In some embodiments, before performing the nucleic acid amplification reaction, prepare a reaction solution respectively in one or more tubes, the reaction solution in each of the tubes including the DNA sample and one of the plurality of probes.

In some embodiments, before performing the nucleic acid amplification reaction, prepare a reaction solution in at least one tube, the reaction solution in each of the at least one tube including the DNA sample and at least two of the plurality of probes.

In some embodiments, performing the nucleic acid amplification reaction using the first probe to the twelfth probe, and prepare reaction solutions in at least two tubes, each of the at least two tubes including one to six of the first to the twelfth probes.

In some embodiments, at least one of the plurality of probes in the same tube has the LNA modification.

In some embodiments, the second probe is not in the same tube as the first probe and the third probe.

In some embodiments, the ninth probe is not in the same tube as the sixth probe, seventh probe, and eighth probe.

In some embodiments, adding the second probe, fourth probe, fifth probe, ninth probe, tenth probe, and twelfth probe to the PCR solution in a first tube; and adding the first probe, third probe, sixth probe, seventh probe, eighth probe, and eleventh probe to the PCR solution in a second tube.

In some embodiments, adding the second probe, fourth probe, fifth probe, and ninth probe to the PCR solution in a first tube; adding the first probe, third probe, sixth probe, seventh probe, and eighth probe to the PCR solution in a second tube; and adding the tenth probe, eleventh probe, and twelfth probe to the PCR solution in a third tube.

### BRIEF DESCRIPTION OF THE DRAWINGS

The present disclosure can be more fully understood by reading the following detailed description of the embodiment, with reference made to the accompanying drawings as follows.

FIG. 1 is a graph showing the amplification curves of fluorescent quantitative PCR according to an example, used to test 4 LNA probes and an MGB probes targeting S531W mutation.

FIG. 2 is a graph showing the amplification curves of fluorescent quantitative PCR according to an example, used to test 3 LNA probes and an MGB probe targeting D516V mutation.

FIG. 3 is a graph showing the amplification curves of fluorescent quantitative PCR according to an example, used to test 3 LNA probes and an MGB probe targeting H526D mutation.

FIG. 4 is a graph showing the amplification curves of fluorescent quantitative PCR according to an example, used to test an LNA probe and an MGB probe targeting H526L mutation.

FIG. 5 is a graph showing the amplification curves of fluorescent quantitative PCR according to an example, used to test an LNA probe and an MGB probe targeting S531L mutation.

FIGS. 6A and 6B are graphs showing the amplification curves of fluorescent quantitative PCR according to an example, used to test the detection effect of adding two probes in one reaction tube.

FIG. 7 is a graph showing the amplification curves of fluorescent quantitative PCR according to an example, used to test the detection effect of adding three probes in one reaction tube.

FIGS. 8A to 8D are graphs showing the amplification curves of fluorescent quantitative PCR according to an example, used to test the detection sensitivity of adding three probes in one reaction tube.

FIGS. 9A to 9E are graphs showing the amplification curves of fluorescent quantitative PCR according to an example, used to compare the detection effects of the MGB probe group and the LNA probe group on different mutation template plasmids.

### DETAILED DESCRIPTION

The following will use drawings and detailed descriptions to clearly illustrate the spirit of the present disclosure. It should be understood that the present disclosure can have various modifications in different aspects, but these modifications do not depart from the scope of the present disclosure, and the descriptions and accompanying drawings are for illustrative purposes and are not intended to limit the present disclosure.

Previous studies have shown that rifampicin resistance of Mycobacterium tuberculosis is typically caused by mutations in rpoB gene, which encodes the β-subunit of RNA polymerase. More than 90% of these mutations are located within an 81-bp region known as the Rifampicin Resistant Determination Region (RRDR). Within this 81bp RRDR, there are multiple mutation sites, including codons 511, 513, 516, 526, 531, and 533, which cover about 98% of clinical rifampicin resistance cases. Most rifampicin resistance mutations of Mycobacterium tuberculosis are single nucleotide polymorphisms (SNPs), and the identification of SNPs requires probes with high specificity. Moreover, in clinical samples (e.g., sputum or culture samples from patients), there is a large amount of wild-type template, while the template having a mutation site is usually very low in abundance. Therefore, the detection probes and the reaction system need to possess high sensitivity.

In embodiments of the present disclosure, probes for nucleic acid amplification reactions are designed based on mutant templates. The biggest advantage of this method is that the presence of a drug resistance mutation site can be directly determined based on the detection results.

Due to the need for sensitivity and/or the high sensitivity requirements in multiplex detection, embodiments of the present disclosure propose using a probe combination containing locked nucleic acids (LNA) for nucleic acid amplification reactions, such as real-time PCR, for detection of rifampicin resistance gene mutation.

LNA is a nucleotide analog in which the ribose ring is “locked” by a methylene bridge connecting the 2′-O atom and the 4′-C atom. LNA monomers include the same nucleic acid bases found in DNA and RNA and can form base pairs according to Watson-Crick base pairing principles. When LNA is incorporated into DNA or RNA oligonucleotides, LNA accelerates pairing with complementary nucleotide strands and increases the stability of the resulting double-stranded nucleic acid chains. Compared to molecular beacons and MGB probes, LNA probes have better specificity and can more accurately identify sequences having single nucleotide mutation, thereby improving detection sensitivity.

Specifically, in embodiments of the present disclosure, a set of probes is provided to detect gene mutations in the 81 bp RRDR of rpoB gene in Mycobacterium tuberculosis using fluorescent quantitative PCR. In some embodiments, the most frequent mutation sites, such as D516V, H526Y, H526R, and S531L, may be prioritized, so the probe combination may include probes corresponding to these mutation sites.

In some embodiments, probes are designed for 12 major mutation types at codons 511, 513, 516, 526, 531, and 533 to detect the types of clinical rifampicin resistance mutations.

In some embodiments, by designing probes corresponding to multiple mutation types (e.g., 2 to 12 types) and through sequence optimization and combination, it is possible to achieve highly sensitive detection of drug-resistant gene mutations. In some embodiments, multiple types of drug resistance gene mutations can be detected simultaneously in the same reaction tube with high sensitivity.

In some embodiments, a kit for detecting rifampicin resistance of Mycobacterium tuberculosis is provided, the kit includes a nucleic acid probe combination including a plurality of probes. Each of these probes is fully complementary to at least one corresponding sequence of various resistance mutation types, and among these probes, at least one probe has an LNA modification. In some embodiments, at least two probes have LNA modifications. In some embodiments, all probes have LNA modifications. In some embodiments, other probes in the nucleic acid probe combination that do not have LNA modifications may have MGB modifications. In other embodiments, other probes in the nucleic acid probe combination that do not have LNA may be Taqman probes. Since LNA-modified probes are more expensive, some probes may have LNA modifications while other probes may have MGB modifications or may be Taqmen probes to meet the sensitivity requirements of the detection.

Table 1 below lists the six major mutation sites of rifampicin resistance and the corresponding mutation types in Mycobacterium tuberculosis, and the names of the corresponding nucleic acid probes in the embodiments of the present disclosure.

TABLE 1

| Codon of mutation site | Mutation type | Probe Name |
|---|---|---|
| Codon 511 | L511P | First probe |
| Codon 513 | Q513K | Second probe |
| | Q513P | Third probe |
| Codon 516 | D516V | Fourth probe |
| | D516Y | Fifth probe |
| Codon 526 | H526D | Sixth probe |
| | H526L | Seventh Probe |
| | H526R | Eighth Probe |
| | H526Y | Ninth Probe |
| Codon 531 | S531L | Tenth Probe |
| | S531W | Eleventh Probe |
| Codon 533 | L533P | Twelfth Probe |

In some embodiments, each of the sequences of these probes corresponding to a mutation type is designed, and each of the probe sequences is completely complementary to the mutation site and the sites near the mutation site.

Table 2 below lists the probe names, the probe codes, the probe sequences, and the sequence identification numbers (SEQ ID number) for the multiple mutation sites. The bold text shows the codon sequence corresponding to the mutation site, and the italic text corresponds to the specific mutation site.

TABLE 2

| | Probe | | SEQ ID |
|---|---|---|---|
| Probe Name | code | Probe sequence | number |
| First probe | M1-1 | CAGCCGAGCCAAT | SEQ ID NO: 1 |
| First probe | M1-2 | CACCAGCCGAGCCAATTC | SEQ ID NO: 2 |
| Second probe | M2-1 | CTGAGCAAATTCATG | SEQ ID NO: 3 |
| Second probe | M2-2 | CAGCTGAGCAAATTCATGGA | SEQ ID NO: 4 |
| Third probe | M3-1 | CTGAGCCCATTCAT | SEQ ID NO: 5 |
| Third probe | M3-2 | CAGCTGAGCCCATTCATGG | SEQ ID NO: 6 |
| Fourth probe | M4-1 | AATTCATGGTCCAGAACA | SEQ ID NO: 7 |
| Fourth probe | M4-2 | GCCAATTCATGGTCCAGAACAAC | SEQ ID NO: 8 |
| Fifth probe | M5-1 | TTCATGTACCAGAACAA | SEQ ID NO: 9 |
| Fifth probe | M5-2 | CAATTCATGTACCAGAACAACCC | SEQ ID NO: 10 |
| Sixth probe | M6-1 | CGCTTGTCGGTCAA | SEQ ID NO: 11 |
| Sixth probe | M6-2 | CGGCGCTTGTCGGTCAACC | SEQ ID NO: 12 |
| Seventh Probe | M7-1 | CTTGAGGGTCAACC | SEQ ID NO: 13 |
| Seventh Probe | M7-2 | GCGCTTGAGGGTCAACCCC | SEQ ID NO: 14 |
| Eighth Probe | M8-1 | CTTGCGGGTCAAC | SEQ ID NO: 15 |
| Eighth Probe | M8-2 | GCGCTTGCGGGTCAACCC | SEQ ID NO: 16 |
| Ninth Probe | M9-1 | CTTGTAGGTCAACCC | SEQ ID NO: 17 |
| Ninth Probe | M9-2 | GCGCTTGTAGGTCAACCCCG | SEQ ID NO: 18 |
| Tenth Probe | M10-1 | CCGACTGTTGGCG | SEQ ID NO: 19 |
| Tenth Probe | M10-2 | GCGCCGACTGTTGGCGCT | SEQ ID NO: 20 |
| Eleventh Probe | M11-1 | CGACTGTGGGCGC | SEQ ID NO: 21 |
| Eleventh Probe | M11-2 | CGCCGACTGTGGGCGCTG | SEQ ID NO: 22 |
| Twelfth Probe | M12-1 | ACTGTCGGCGCCGG | SEQ ID NO: 23 |
| Twelfth Probe | M12-2 | CCGACTGTCGGCGCCGGGG | SEQ ID NO: 24 |

In some embodiments, mutation sites with higher occurrence rates are preferentially selected for detection. In some embodiments, it is preferred to use a probe combination covering the six mutation sites for simultaneous detection and use a respective probe for each of the mutation sites. In some embodiments, it is preferred to use 12 probes covering the six mutation sites and corresponding to the 12 mutation types for simultaneous detection, thereby quickly identifying the specific mutation type that leads to drug resistance.

In some embodiments, each of the plurality of probes is a dual-labeled fluorescent probe, including: a fluorescent group at the 5′ end and a quencher group at the 3′ end. The fluorescent group at the 5′ end may be, for example, FAM, Hex, TET, VIC, Cy5, Rox, or the like. The quencher group at the 3′ end may be, for example, BHQ1, BHQ2, BHQ3, or the like.

In some embodiments of the present disclosure, at least one probe in the nucleic acid probe combination is an LNA-modified probe, i.e., a probe having an LNA modification. Based on the characteristics of LNA probes, one or more LNA-modified nucleotide sites can be selectively inserted into the DNA sequence during synthesis, so any one or more nucleotide sites in the probe sequence can be modified with LNAs to obtain different LNA probes. Different numbers of nucleotide modifications in the probe can result in different Tm values, and the recognition ability and affinity of the target sequence will also vary depending on the nucleotide sites modified with LNAs. Therefore, the amplification efficiency of differently designed LNA probes may have significant differences.

In some embodiments, by optimizing the design of LNA probes, high-sensitivity mutation detection probes can be obtained. In some embodiments, the LNA modification is located at the mutation site and adjacent 1 to 2 bases. In some embodiments, the LNA modification includes 3 LNA-modified nucleotides within a range of 2 bases on both sides of a mutated base. In some embodiments, the LNA modification is within a range of 2 bases on both sides of a mutation base and includes two consecutive LNA-modified nucleotides and another LNA-modified nucleotide spaced apart.

In some embodiments, the number of bases in the probe sequence modified with LNAs ranges from 3 to 8, with a preferred range of 4 to 6; and the LNA modification sites are distributed on both sides of the mutated base.

Table 3 below lists the probe sequences having LNA modifications in the embodiments of the present disclosure, including probe names, probe codes, sequence ID numbers, and the number of LNA modification sites (i.e., the number of LNAs). The bold text shows the codon sequence corresponding to the mutation site, the bold italic text corresponds to the specific mutation site, and the site in brackets (i.e., [N], where N represents A, T, C, or G) indicates that this is the site modified with LNA.

TABLE 3

| | | | SEQ | |
|---|---|---|---|---|
| Probe | Probe | | ID | LNA |
| Name | code | Probe sequence | NO: | Number |
| First | LNA-M1-1 | C[A]G[CC]G[A]G[C]CAAT | 25 | 5 |
| probe | | | | |
| First | LNA-M1-2 | CACC[A]G[CC]G[A]G[C]CAATTC | 26 | 5 |
| probe | | | | |
| Second | LNA-M2-1 | C[T]G[A]G[CA]A[A]T[T]CATG | 27 | 6 |
| probe | | | | |
| Second | LNA-M2-2 | CAGC[T]G[A]G[CA]A[A]T[T]CATGGA | 28 | 6 |
| probe | | | | |
| Third | LNA-M3-1 | CT[G]A[G]C[CC]A[T]T[C]AT | 29 | 6 |
| probe | | | | |
| Third | LNA-M3-2 | CAGCT[G]A[G]C[CC]A[T]T[C]ATGG | 30 | 6 |
| probe | | | | |
| Fourth | LNA-M4-1A | AATTCA[T]G[GT]C[C]AGAACA | 31 | 4 |
| probe | | | | |
| Fourth | LNA-M4-1B | AATT[C]A[T]G[GT]C[C]AGAACA | 32 | 5 |
| probe | | | | |
| Fourth | LNA-M4-1C | AATT[C]A[T]G[GT]C[C]A[G]AACA | 33 | 6 |
| probe | | | | |
| Fourth | LNA-M4-2A | GCCAATTCA[T]G[GT]C[C]AGAACAAC | 34 | 4 |
| probe | | | | |
| Fourth | LNA-M4-2B | GCCAATT[C]A[T]G[GT]C[C]AGAACAAC | 35 | 5 |
| probe | | | | |
| Fourth | LNA-M4-2C | GCCAATT[C]A[T]G[GT]C[C]A[G]AACAAC | 36 | 6 |
| probe | | | | |
| Fifth | LNA-M5-1 | T[T]C[A]T[GT]A[C]C[A]GAACAA | 37 | 6 |
| probe | | | | |
| Fifth | LNA-M5-2 | CAAT[T]C[A]T[GT]A[C]C[A]GAACAACCC | 38 | 6 |
| probe | | | | |
| Sixth | LNA-M6-1A | CGCT[T]G[TC]G[G]TCAA | 39 | 4 |
| probe | | | | |
| Sixth | LNA-M6-1B | CG[C]T[T]G[TC]G[G]TCAA | 40 | 5 |
| probe | | | | |
| Sixth | LNA-M6-1C | CG[C]T[T]G[TC]G[G]T[C]AA | 41 | 6 |
| probe | | | | |
| Sixth | LNA-M6-2A | CGGCGCT[T]G[TC]G[G]TCAACC | 42 | 4 |
| probe | | | | |
| Sixth | LNA-M6-2B | CGGCG[C]T[T]G[TC]G[G]TCAACC | 43 | 5 |
| probe | | | | |
| Sixth | LNA-M6-2C | CGGCG[C]T[T]G[TC]G[G]T[C]AACC | 44 | 6 |
| probe | | | | |
| Seventh | LNA-M7-1 | C[T]T[GA]G[G]G[T]C[A]ACC | 45 | 6 |
| Probe | | | | |
| Seventh | LNA-M7-2 | GCGC[T]T[GA]G[G]G[T]C[A]ACCCC | 46 | 6 |
| Probe | | | | |
| Eighth | LNA-M8-1 | C[T]T[GC]G[G]G[T]CAAC | 47 | 5 |
| Probe | | | | |
| Eighth | LNA-M8-2 | GCGC[T]T[GC]G[G]G[T]CAACCC | 48 | 5 |
| Probe | | | | |
| Ninth | LNA-M9-1 | C[T]T[GT]A[G]GTCAACCC | 49 | 4 |
| Probe | | | | |
| Ninth | LNA-M9-2 | GCGC[T]T[GT]A[G]GTCAACCCCG | 50 | 4 |
| Probe | | | | |
| Tenth | LNA-M10-1 | CCG[A]C[T]G[TT]G[G]CG | 51 | 5 |
| Probe | | | | |
| Tenth | LNA-M10-2 | GCGCCG[A]C[T]G[TT]G[G]CGCT | 52 | 5 |
| Probe | | | | |
| Eleventh | LNA-M11-1A | CGAC[T]G[TG]GGCGC | 53 | 3 |
| Probe | | | | |
| Eleventh | LNA-M11-1B | CGAC[T]G[TG]G[G]CGC | 54 | 4 |
| Probe | | | | |
| Eleventh | LNA-M11-1C | CG[A]C[T]G[TG]G[G]CGC | 55 | 5 |
| Probe | | | | |
| Eleventh | LNA-M11-1D | CGA[CT]G[TG]G[GC]GC | 56 | 6 |
| Probe | | | | |
| Eleventh | LNA-M11-2A | CGCCGAC[T]G[TG]GGCGC | 57 | 3 |
| Probe | | | | |
| Eleventh | LNA-M11-2B | CGCCGAC[T]G[TG]G[G]CGC | 58 | 4 |
| Probe | | | | |
| Eleventh | LNA-M11-2C | CGCCG[A]C[T]G[TG]G[G]CGCTG | 59 | 5 |
| Probe | | | | |
| Eleventh | LNA-M11-2D | CGCGA[CT]G[TG]G[GC]GCTG | 60 | 6 |
| Probe | | | | |
| Twelfth | LNA-M12-1 | ACTGTCGG[C]G[CC]G[G] | 61 | 4 |
| Probe | | | | |
| Twelfth | LNA-M12-2 | CCGACTGTCGG[C]G[CC]G[G]GG | 62 | 4 |
| Probe | | | | |

In some embodiments, during detection, since the sample contains a large amount of wild-type template, a probe completely matching the wild-type template corresponding to the mutation site, also known as a blocker probe, is added to the reaction solution to block the amplification reaction that may be caused by the wild-type template. Therefore, the addition of the blocker probe reduces false positive signals and improves detection specificity. In some embodiments, a plurality of blocker probes are added in a reaction solution. In some embodiments, the blocker probe used may be, for example, a peptide nucleic acid (PNA) probe or an MGB probe.

The following provides experimental examples to illustrate the detection capabilities of the nucleic acid probes and nucleic acid probe combinations in the embodiments of the present disclosure. In the examples, the amplification reaction was performed using nested PCR, wherein the outer primer pair is Nest PF and Nest PR, and the inner primer pair is RpoB PF and RpoB PR. The sequences of the primer pairs in the examples are shown in Table 4 below.

TABLE 4
Primer sequences

| Primer | | |
|---|---|---|
| Name | Sequence | SEQ ID number |
| Nest PF | AGGCGATCACACCGCAGACG | SEQ ID NO: 63 |
| Nest PR | CCGACAGCGAGCCGATCAGA | SEQ ID NO: 64 |
| RpoB PF | CATCCGGCCGGTGGT | SEQ ID NO: 65 |
| RpoB PR | GGCACGCTCACGTGACAG | SEQ ID NO: 66 |

### 1. Design and Optimization of LNA Probe Sequences

### 1.1 Optimization Example of LNA-Modified Probe for Detecting S531W Mutation (Eleventh Probe)

Taking the eleventh probe M11-1 (SEQ ID NO: 21) for detecting S531W mutation as an example, different LNA-modified probes were tested to illustrate the effect of the number and positions of LNA modification sites on detection sensitivity. By synthesizing probe M11-1 with different LNA modifications, four LNA-modified probes were obtained: probe LNA-M11-1A, probe LNA-M11-1B, probe LNA-M11-1C, and probe LNA-M11-1D. Additionally, an MGB-modified probe, probe MGB-M11-1, was synthesized, with a FAM fluorescent group at the 5′ end and an MGB group at the 3′ end. Table 5 lists the sequences, number of LNA modification sites, and LNA modification sites (i.e. LNA sites) of the four LNA probes and the MGB probe. The bold text shows the codon sequence corresponding to the mutation site, the bold italic text corresponds to the specific mutation site, and [N] indicates the site modified with LNA.

TABLE 5

| | | SEQ ID | Number | |
|---|---|---|---|---|
| Probe code | Probe Sequence (5′ > 3′) | number | of LNAs | LNA site |
| LNA-M11-1A | CGAC[T]G[TG]GGCGC | SEQ ID | 3 | 5,7,8 |
| | | NO: 53 | | |
| LNA-M11-1B | CGAC[T]G[TG]G[G]CGC | SEQ ID | 4 | 5, 7, 8, 10 |
| | | NO: 54 | | |
| LNA-M11-1C | CG[A]C[T]G[TG]G[G]CGC | SEQ ID | 5 | 3, 5, 7, 8, |
| | | NO: 55 | | 10 |
| LNA-M11-1D | CGA[CT]G[TG]G[GC]GC | SEQ ID | 6 | 4, 5, 7, 8, |
| | | NO: 56 | | 10, 11 |
| MGB-M11-1 | CGACTGTGGGCGC | SEQ ID | 0 | none |
| | | NO: 21 | | |

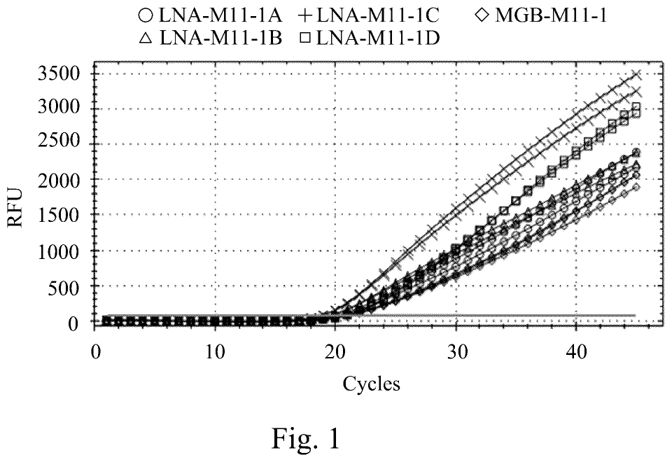

The four LNA probes and the MGB probe for detecting S531W mutant were tested using fluorescent quantitative PCR according to the single PCR reaction mode (i.e., one probe added in a PCR tube) in Table 6 and the PCR reaction procedure in Table 7, using Biorad CFX96 Real-time PCR Detection system. The results are shown in FIG. 1.

TABLE 6
Single PCR reaction mode

| Component | Volume (μl) | Final concentration | |
|---|---|---|---|
| QuantiNova Reaction Mix | 13.5 | | |
| Primer Nest PF (50 μM) | 0.3 | 600 | nM |
| Primer Nest PR (50 μM) | 0.3 | 600 | nM |
| Primer RpoB PF (50 μM) | 0.3 | 600 | nM |
| Primer RpoB PR (50 μM) | 0.3 | 600 | nM |
| MGB probe or LNA probe (2 μM) | 5 | 400 | nM |
| Plasmid (about 1000 copies/μl) | 1 | 1000 | copies |

| | | |
|---|---|---|
| ddH2O | 4.3 | — |
| Total volume | 25 | — |

TABLE 7
Fluorescence quantitative PCR amplification procedure

| | | |
|---|---|---|
| temperature | time | Number of cycles |

| | | | |
|---|---|---|---|
| 95° C. | 2 | minutes | 1 |
| 95° C. | 15 | seconds | 10 |
| 65° C. | 30 | seconds | |
| 95° C. | 15 | seconds | 45 |
| 75° C. | 15 | seconds | |
| 60° C. | 15 | seconds | |

Refer to FIG. 1 and Table 8, it can be seen that under the single PCR detection mode, compared with the MGB probe, the Cq values of the four LNA probes were all significantly earlier, and the relative fluorescence unity (RFU) values at the amplification endpoint of the four LNA probes were also significantly increased. Further, probe LNA-M11-1C had the best amplification effect, showing a significant improvement in detection sensitivity relative to the MGB probe. The corresponding Cq values and endpoint RFU values are shown in Table 8. The negative control (NTC) group used water instead of the mutation template plasmid. The above results indicate that different nucleotide modification sites in the probe can result in LNA probes with different amplification efficiencies. Through screening and optimization, high-sensitivity LNA probes that meet the needs can be obtained.

TABLE 8
Amplification results of different LNA probes
and MGB probe for detecting S531W mutation

| | | Cq-value | | | |
|---|---|---|---|---|---|
| Probe code | FAM-Cq value | average | RFU | ΔC | ΔRFU |
| MGB-M11-1 | 21.5 | 21.47 | 2000 | 0 | 0 |
| | 21.64 | | | | |
| | 21.28 | | | | |
| LNA-M11-1A | 20.73 | 20.58 | 2500 | −0.89 | 500 |
| | 20.43 | | | | |
| LNA-M11-1B | 19.85 | 19.85 | 2500 | −1.62 | 500 |
| | 19.85 | | | | |
| LNA-M11-1C | 19.11 | 19.14 | 3500 | −2.33 | 1500 |
| | 19.17 | | | | |
| LNA-M11-1D | 21.18 | 21.21 | 3000 | −0.27 | 1000 |
| | 21.23 | | | | |
| NTC | NA | — | — | — | — |

### 1.2 Optimization Example of LNA-Modified Probe for Detecting D516V Mutation (Fourth Probe)

Taking the fourth probe M4-1 (SEQ ID NO: 7) for detecting D516V mutation as an example, different LNA-modified probes were tested to illustrate the effect of the number and positions of LNA modification sites on detection sensitivity. By synthesizing M4-1 with different LNA modifications, three LNA-modified probes were obtained: probe LNA-M4-1A, probe LNA-M4-1B, and probe LNA-M4-1C. Additionally, an MGB-modified probe, probe MGB-M4-1, was synthesized, with a FAM fluorescent group at the 5′ end and an MGB group at the 3′ end. Table 9 lists the sequences, number of LNA modification sites, and LNA modification sites of the three LNA probes and the MGB probe. The bold text shows the codon sequence corresponding to the mutation site, the bold italic text corresponds to the specific mutation site, and [N] indicates the site modified with LNA.

TABLE 9

| | | SEQ ID | Number | LNA |
|---|---|---|---|---|
| Probe code | Probe Sequence (5′ > 3′) | number | of LNAs | site |
| LNA-M4-1A | AATTCA[T]G[GT]C[C]AGAACA | SEQ ID | 4 | 7, 9, |
| | | NO: 31 | | 10, 12 |
| LNA-M4-1B | AATT[C]A[T]G[GT]C[C]AGAACA | SEQ ID | 5 | 5, 7, 9, |
| | | NO: 32 | | 10, 12 |
| LNA-M4-1C | AATT[C]A[T]G[GT]C[C]A[G]AACA | SEQ ID | 6 | 5, 7, 9, |
| | | NO: 33 | | 10, |
| | | | | 12, 14 |
| MGB-M4-1 | AATTCATGGTCCAGAACA | SEQ ID | 0 | None |
| | | NO: 7 | | |

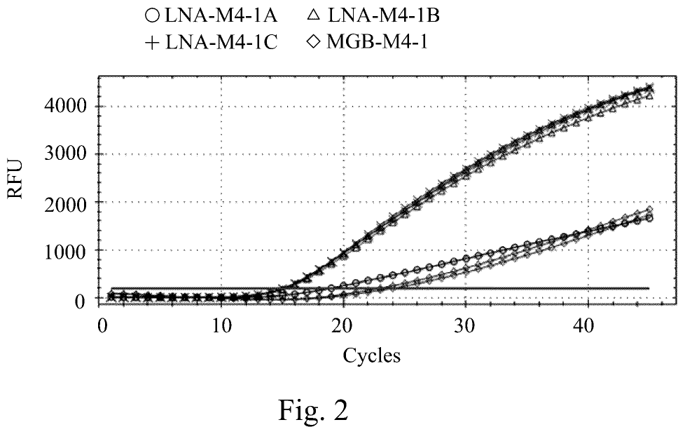

The three LNA probes and the MGB probe for detecting D516V mutation were tested according to the single PCR reaction mode in Table 6 and the PCR reaction program in Table 7 using Biorad CFX96 Real-time PCR Detection system. The results are shown in FIG. 2.

Refer to FIG. 2 and Table 10, it can be seen that under the single PCR detection mode, compared with the MGB probe, the Cq values of the three LNA probes were all significantly earlier, and the relative fluorescence unity (RFU) values at the amplification endpoint of probe LNA-M4-1B and probe LNA-M4-1C were also significantly increased. The corresponding Cq values and endpoint RFU values for the amplification reaction are listed in Table 10.

TABLE 10
Amplification results of different LNA probes
and MGB probe for detecting D516V mutation

| | | Average | | | |
|---|---|---|---|---|---|
| | | Cq | | | |
| Probe | FAM-Cq value | value | RFU | ΔC | ΔRFU |
| MGB-M4-1 | 23.67 | 23.16 | 1700 | 0 | 0 |
| | 22.64 | | | | |
| LNA-M4-1A | 18.64 | 18.61 | 1700 | −4.56 | 0 |
| | 18.57 | | | | |
| LNA-M4-1B | 15.03 | 15.10 | 4100 | −8.06 | 2400 |
| | 15.17 | | | | |
| LNA-M4-1C | 14.86 | 14.84 | 4100 | −8.32 | 2400 |
| | 14.82 | | | | |
| NTC | NA | — | — | — | — |
| | NA | | | | |

### 1.2 Optimization Example of LNA-Modified Probe for Detecting H526D Mutation (Sixth Probe)

Taking the sixth probe M6-1 (SEQ ID NO: 11) for detecting H526D mutation as an example, different LNA-modified probes were tested to illustrate the effect of the number and positions of LNA modification sites on detection sensitivity. By synthesizing probe M6-1 with different LNA modification sites, three LNA-modified probes were obtained: probe LNA-M6-1A, probe LNA-M6-1B, and probe LNA-M6-1C. Additionally, an MGB-modified probe, probe MGB-M6-1, was synthesized, with a FAM fluorescent group at the 5′ end and an MGB group at the 3′ end. Table 11 lists the sequences, the number of LNA modifications sites, and LNA modification sites of the three LNA probes and the MGB probe. The bold text shows to the codon sequence corresponding to the mutation site, the bold italic text corresponds to the specific mutation site, and [N] indicates the site modified with LNA.

TABLE 11

| | | SEQ ID | Number | |
|---|---|---|---|---|
| Probe Code | Probe Sequence (5′ > 3′) | number | of LNAs | LNA site |
| LNA-M6-1A | CGCT[T]G[TC]G[G]TCAA | SEQ ID NO: | 4 | 5, 7, 8, 10 |
| | | 39 | | |
| LNA-M6-1B | CG[C]T[T]G[TC]G[G]TCAA | SEQ ID NO: | 5 | 3, 5, 7, 8, |
| | | 40 | | 10 |
| LNA-M6-1C | CG[C]T[T]G[TC]G[G]T[C]AA | SEQ ID NO: | 6 | 3, 5, 7, 8, |
| | | 41 | | 10, 12 |
| MGB-M6-1 | CGCTTGTCGGTCAA | SEQ ID NO: | 0 | none |
| | | 11 | | |

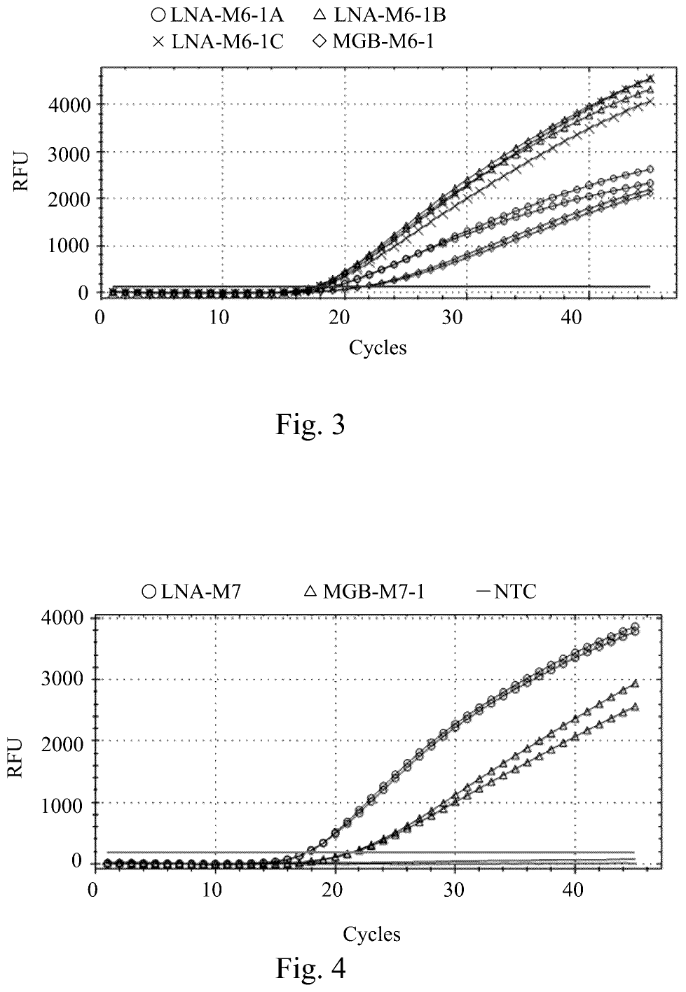

The three LNA probes and the MGB probe for detecting H526D were tested according to the single PCR reaction mode in Table 6 and the PCR reaction procedure in Table 7, using Biorad CFX96 Real-time PCR Detection system. The results are shown in FIG. 3.

Refer to FIG. 3 and Table 12, it can be seen that under the single PCR detection mode, compared with the MGB probe, the Cq values of the three LNA probes were all significantly earlier, and the RFU values at the amplification endpoint were also significantly increased. The corresponding Cq values and endpoint RFU values for the amplification reaction are listed in Table 12.

TABLE 12
Amplification results of different LNA probes
and MGB probe for detecting H526D mutation

| | | Average | | | |
|---|---|---|---|---|---|
| Probe | FAM-Cq value | Cq value | RFU | ΔC | ΔRFU |
| MGB-M6-1 | 22.84 | 22.67 | 2000 | — | — |
| | 22.49 | | | | |
| LNA-M6-1A | 19.73 | 19.78 | 2500 | −2.90 | 500 |
| | 19.82 | | | | |
| LNA-M6-1B | 18.08 | 18.10 | 4500 | −4.57 | 2500 |
| | 18.12 | | | | |
| LNA-M6-1C | 18.37 | 18.48 | 4500 | −4.19 | 2500 |
| | 18.59 | | | | |
| NTC | NA | — | — | — | — |
| | NA | | | | |

### 2. 2. Testing the Amplification Effect of LNA Probes in Single PCR Reaction Mode

By optimizing the modification scheme of LNA probes, high-sensitivity LNA probes can be obtained, and the amplification effect can be better than that of MGB probes. The optimized LNA probes LNA-M7-1 and LNA-M10-1 are used as examples to illustrate this. The LNA probes LNA-M7-1 and LNA-M10-1 were tested to detect the corresponding mutation templates H526L and S531L, respectively, and were respectively compared with corresponding MGB probes. The corresponding MGB probes are MGB-M7-1 and MGB-M10-1, with a FAM fluorescent group at the 5′ end and an MGB group at the 3′ end. Table 13 lists the sequences and sequence numbers of the probes. The bold text shows the codon sequence corresponding to the mutation site, the bold italic text corresponds to the specific mutation site, and [N] indicates the site modified with LNA.

TABLE 13
Probe sequences for detecting H526L mutation and S531L
mutation

| | Mutation | | |
|---|---|---|---|
| Probe | type | Probe Sequence (5′ > 3′) | SEQ ID number |
| LNA-M7-1 | H526L | C[T][GA]G[G]G[T]C[A]ACC | SEQ ID NO: 45 |
| MGB-M7-1 | H526L | CTTGAGGGTCAACC | SEQ ID NO: 13 |
| LNA-M10-1 | S531L | CCG[A]C[T]G[TT]G[G]CG | SEQ ID NO: 51 |
| MGB-M10-1 | S531L | CCGACTGTTGGCG | SEQ ID NO: 19 |

According to the single PCR reaction mode in Table 6, reaction tubes each containing one of the three different LNA probes and the MGB probe were prepared, with the mutation template plasmid added at 1000 copies. The amplification reactions were performed according to the PCR reaction procedure in Table 7, using Biorad CFX96 Real-time PCR Detection system.

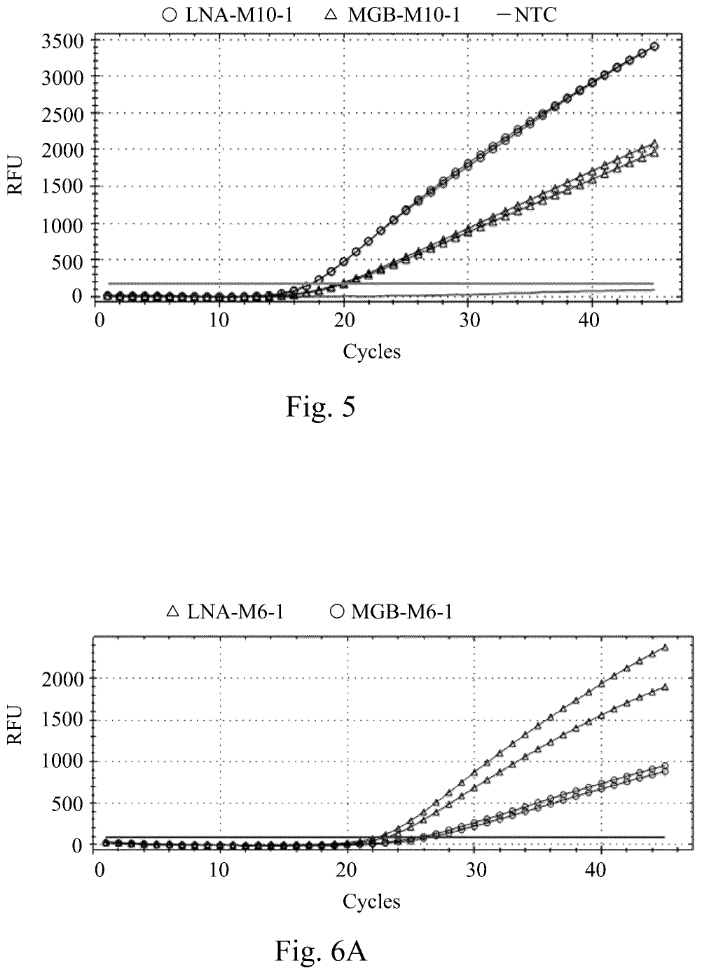

The amplification results of LNA probe LNA-M7-1 and MGB probe MGB-M7-1 are shown in FIG. 4 and Table 14. The amplification results of the LNA probe LNA-M10-1 and the MGB probe MGB-M10-1 are shown in FIG. 5 and Table 15. The amplification results show that the optimized LNA probes for H526L and S531L mutation templates had earlier Cq values and significantly higher fluorescence intensity compared to the MGB probes, further enhancing the detection sensitivity.

TABLE 14
Amplification effects of the LNA probe and
the MGB probe for detecting H526L mutation

| | FAM-Cq | Average | | | |
|---|---|---|---|---|---|
| Probe | value | Cq value | RFU | ΔCq | ΔRFU |
| MGB-M7-1 | 21.53 | 21.44 | 2700 | — | — |
| | 21.35 | | | | |
| | 18.11 | | | | |
| LNA-M7-1 | 17.65 | 17.69 | 3900 | −3.75 | 1200 |
| | 17.73 | | | | |

TABLE 15
Amplification effects of the LNA probe and
the MGB probe for detecting S531L mutation

| | FAM-Cq | Average | | | |
|---|---|---|---|---|---|
| Probe | value | Cq value | RFU | ΔCq | ΔRFU |
| MGB-M10-1 | 20.09 | 19.92 | 2000 | — | — |
| | 19.74 | | | | |
| LNA-M10-1 | 17.4 | 17.40 | 3500 | −2.53 | 1500 |
| | 17.39 | | | | |

### 3. 3. Amplification Effect of LNA Probes in Multiplex PCR Reaction Mode

Since rifampicin resistance-related mutation sites of Mycobacterium tuberculosis are distributed in multiple positions within the 81bp region of rpoB gene, it is desired to detect as many mutation sites as possible by using multiple probes in a PCR solution for multiplex amplification. This allows for the simultaneous detection of multiple mutation sites in the same PCR tube. Such a multiplex reaction mode can reduce the number of PCR reaction tubes and the reagent amount, shorten the overall detection time, and improve detection efficiency. When multiple LNA probes are placed in the same tube, the LNA probes need to maintain high amplification performance in the complex amplification system to meet clinical detection requirements.

### 3.1 Sensitivity Evaluation of LNA Probes in Multiplex PCR Detection

### 3.1.1 Dual Detection of H526D and H526L Mutations

In one reaction tube, two mutation types, H526D and H526L, were detected simultaneously by adding the corresponding two probes: probe LNA-M6-1 for H526D mutation and probe LNA-M7-1 probe for H526L mutation. In another tube, two MGB probes with the same probe sequences, probe MGB-M6-1 and probe MGB-M7-1, were tested and serve as a control group. The probe sequences are shown in Table 16. The bold text shows the codon sequence corresponding to the mutation site, the bold italic text corresponds to the specific mutation site, and [N] indicates the site modified with LNA.

TABLE 16
Probe sequences for dual PCR detection

| Probe code | Mutation type | Probe Sequence (5′ > 3′) | SEQ ID number |
|---|---|---|---|
| LNA-M6-1B | H526D | CG[C]T[T]G[TC]G[G]TCAA | SEQ ID NO: 40 |
| LNA-M7-1 | H526L | C[T]T[GA]G[G]G[T]C[A]ACC | SEQ ID NO: 45 |
| MGB-M6-1 | H526D | CGCTTGTCGGTCAA | SEQ ID NO: 11 |
| MGB-M7-1 | H526L | CTTGAGGGTCAACC | SEQ ID NO: 13 |

The reaction solutions were prepared according to Table 17, and 1000 copies of H526D and H526L mutation template plasmids were added. In the control group, the LNA probes LNA-M6-1 and LNA-M7-1 were replaced with the MGB probes MGB-M6-1 and MGB-M7-1. The amplification reactions were performed using the PCR procedure in Table 7, using Biorad CFX96 Real-time PCR Detection system.

TABLE 17
Dual amplification mode for detecting
H526D and H526L mutations

| | | Final | |
|---|---|---|---|
| Component | Volume (μl) | concentration | |
| QuantiNova Reaction Mix | 13.5 | | |
| Primer Nest PF (50 μM) | 0.3 | 600 | nM |
| Primer Nest PR (50 μM) | 0.3 | 600 | nM |
| Primer RpoB PF (50 μM) | 0.3 | 600 | nM |
| Primer RpoB PR (50 μM) | 0.3 | 600 | nM |
| LNA-M6-1 (2 μM) | 2.5 | 200 | nM |
| LNA-M7-1 (2 μM) | 2.5 | 200 | nM |
| PNAs 511 + 513 (10 μM) | 0.2 | 400 | nM |
| Internal control primer PF (10 μM) | 0.375 | 150 | nM |
| Internal control primer PR (10 μM) | 0.375 | 150 | nM |
| Internal control probe (10 μM) | 0.1875 | 75 | nM |
| Internal probe template | 0.15 | 75 | copies |
| DNA (500 copies/μl) | | | |

| | | |
|---|---|---|
| Mutation template plasmid | 1 | |
| dH₂O | Add to 25 μl | |
| Total volume | 25 | — |

PNAs 511 and 513 are blocker probes used to block the amplification of wild-type template at codons 511 and 513.

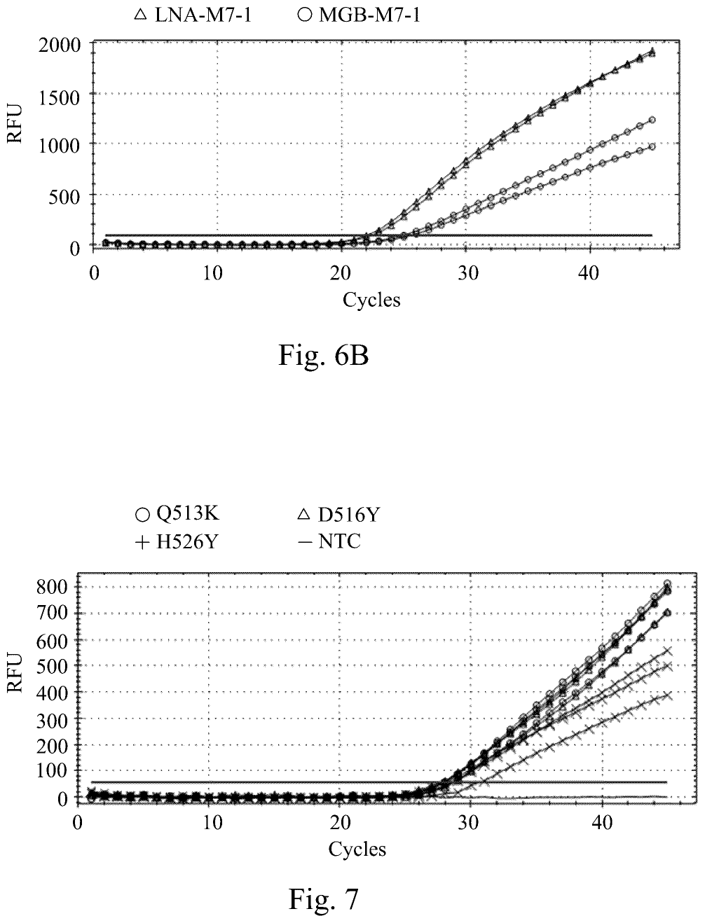

The amplification curves are shown in FIGS. 6A and 6B, and the amplification Cq values are shown in Table 18. The amplification results show that when H526D and H526L mutations were detected in the same tube, the LNA probes had higher endpoint fluorescence brightness and significantly earlier Cq values, indicating that the LNA probes designed in this application can effectively improve the amplification effect and increase the detection sensitivity in dual detection.

TABLE 18
Results of dual amplification mode for detecting
H526D mutation and H526L mutation

| Mutation | MGB Probe Set | | | LNA Probe Set | | |
|---|---|---|---|---|---|---|
| template | FAM-Cq | Average | | FAM-Cq | Average | |
| plasmid | value | Cq value | RFU | value | Cq value | RFU |
| H526D | 26.27 | 25.90 | 1000 | 23.01 | 22.60 | 2000 |
| | 25.52 | | | 22.19 | | |
| H526L | 25.39 | 25.13 | 1000 | 21.91 | 22.12 | 2000 |
| | 24.86 | | | 22.33 | | |

### 3.1.2 Multiplex Detection of Q513K, D516Y, and H526Y Mutations

In one tube, three mutation types, Q513K, D516Y, and H526Y, were detected simultaneously by adding the corresponding three LNA probes: probe LNA-M2-1 for Q513K mutation, probe LNA-M5-1 for D516Y mutation, and probe LNA-M9-1 for H526Y mutation. The probe sequences are shown in Table 19. The bold text shows the codon sequence corresponding to the mutation site, the bold italic text corresponds to the specific mutation site, and [N] indicates the site modified with LNA.

TABLE 19
Probe sequences for Q513K, D516Y, and H526Y mutations

| | | | SEQ ID |
|---|---|---|---|
| Probe code | Mutation type | LNA Probe Sequence (5′ > 3′) | number |
| LNA-M2-1 | Q513K | C[T]G[A]G[CA]A[A]T[T]CATG | SEQ ID NO: |
| | | | 27 |
| LNA-M5-1 | D516Y | T[T]C[A]T[GT]A[C]C[A]GAACAA | SEQ ID NO: |
| | | | 37 |
| LNA-M9-1 | H526Y | C[T]T[GT]A[G]GTCAACCC | SEQ ID NO: |
| | | | 49 |

According to Table 20, the reaction system was prepared, and 10 copies of the Q513K, D516Y, and H526Y mutation plasmids were added. The PCR reaction program in Table 7 was followed using the Biorad CFX96 real-time PCR instrument for amplification. The reaction solutions were prepared according to Table 20, and 10 copies of three mutation template plasmids, Q513K mutation, D516Y mutation and H526Y mutation, were added respectively. The amplification reaction was performed according to the PCR procedure in Table 7, using Biorad CFX96 Real-time PCR Detection system.

TABLE 20
Multiplex PCR reaction mode for Q513K mutation,
D516Y mutation, and H526Y mutation

| | | Final | |
|---|---|---|---|
| Component | Volume (μl) | concentration | |
| QuantiNova Reaction Mix | 13.5 | | |
| Primer Nest PF (50 μM) | 0.3 | 600 | nM |
| Primer Nest PR (50 μM) | 0.3 | 600 | nM |
| Primer RpoB PF (50 μM) | 0.3 | 600 | nM |
| Primer RpoB PR (50 μM) | 0.3 | 600 | nM |
| LNA-M2-1 (10 μM) | 0.25 | 100 | nM |
| LNA-M5-1 (10 μM) | 0.25 | 100 | nM |
| LNA-M9-1 (10 μM) | 0.25 | 100 | nM |
| PNA 516 (10 μM) | 1 | 400 | nM |
| PNA 526 (10 μM) | 1 | 400 | nM |
| Internal control primer PF (10 μM) | 0.375 | 150 | nM |
| Internal control primer PR (10 μM) | 0.375 | 150 | nM |
| Internal control probe (10 μM) | 0.1875 | 75 | nM |
| Internal control template | 0.15 | 75 | copies |

| | | |
|---|---|---|
| DNA (500 copies/μl) | | |
| Mutation template plasmid | 1 | |
| dH₂O | Add to 25 | |
| Total volume | 25 | — |

PNA 516 is a blocker probe used to block the amplification of the wild-type template at codon 516, and PNA 526 is a blocker probe used to block the amplification of the wild-type template at codon 526.

The amplification curves are shown in FIG. 7, and the amplification Cq values are shown in Table 21. From the results of the amplification, it can be seen that in the multiplex detection of the three mutations Q513K, D516Y, and H526Y, all three repeated PCRs can stably amplify when 10 copies of the mutation template plasmids were added. This indicates that in the multiplex detection, the LNA probes designed in this application can achieve relatively stable amplification results even with a low copy number of mutation template plasmids, demonstrating the higher detection sensitivity of the probe design in embodiments of the present disclosure.

TABLE 21
Results of multiplex amplification mode for
detecting Q513K, D516Y, and H526Y mutations

| Mutation template | FAM-Cq | Average Cq | |
|---|---|---|---|
| plasmid | value | value | RFU |
| Q513K | 29.78 | 29.33 | 700 |
| | 29.14 | | |
| | 29.07 | | |
| D516Y | 29.78 | 29.33 | 700 |
| | 29.14 | | |
| | 29.07 | | |
| H526Y | 29.49 | 30.48 | 500 |
| | 32.09 | | |
| | 29.85 | | |

### 3.1.3 Multiplex PCR Mode for Detecting S531L, S531W, and L533P Mutations

Taking the mutant detection probe LNA-M11-1 as an example, the performance of the LNA probes in multiplex detection is illustrated. In one PCR tube, three mutation types, S531L, S531W, and L533P, were detected simultaneously by adding the corresponding three probes: probe MGB-M10-1 for S531L mutation, probe LNA-M11-1C or probe MGB-M11-1 for S531W mutation, and probe MGB-M12 for L533P mutation. The probe sequences are shown in Table 22. The bold text shows the codon sequence corresponding to the mutation site, the bold italic text corresponds to the specific mutation site, and [N] indicates the site modified with LNA.

TABLE 22
Probe sequences for multiplex detection of S531L, S531W,
and L533P mutations

| | | Nucleic acid probe | SEQ ID |
|---|---|---|---|
| Probe code | Mutation type | sequence (5′ > 3′) | number |
| MGB-M10-1 | S531L | CCGACTGTTGGCG | SEQ ID NO: |
| | | | 19 |
| LNA-M11-1C | S531W | CG[A]C[T]G[TG]G[G]CGC | SEQ ID NO: |
| | | | 55 |
| MGB-M11-1 | S531W | CGACTGTGGGCGC | SEQ ID NO: |
| | | | 21 |
| MGB-M12-1 | L533P | ACTGTCGGCGCCGG | SEQ ID NO: |
| | | | 23 |

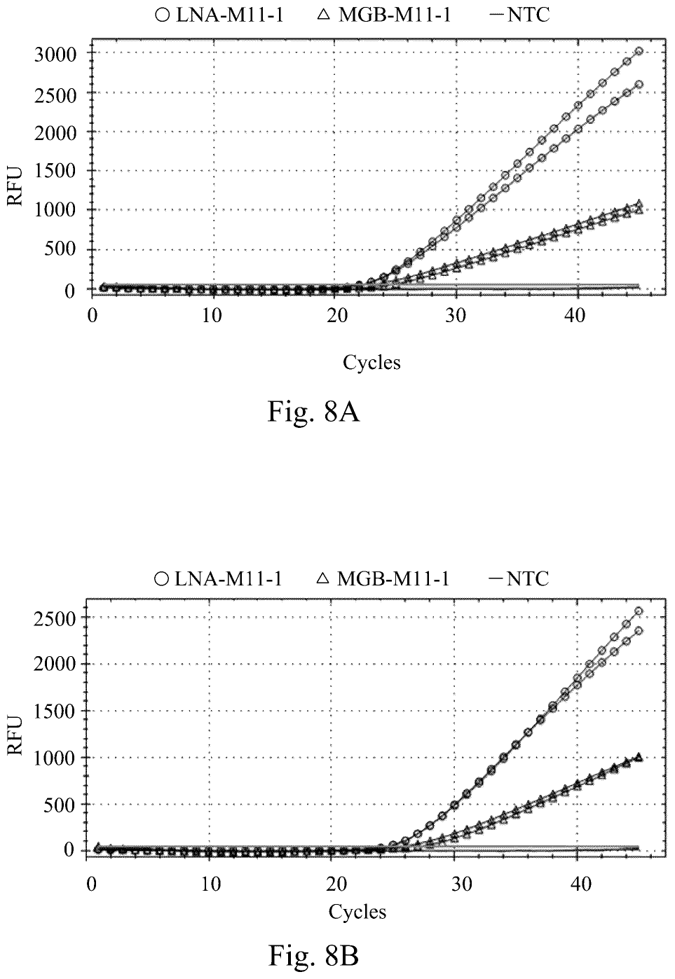

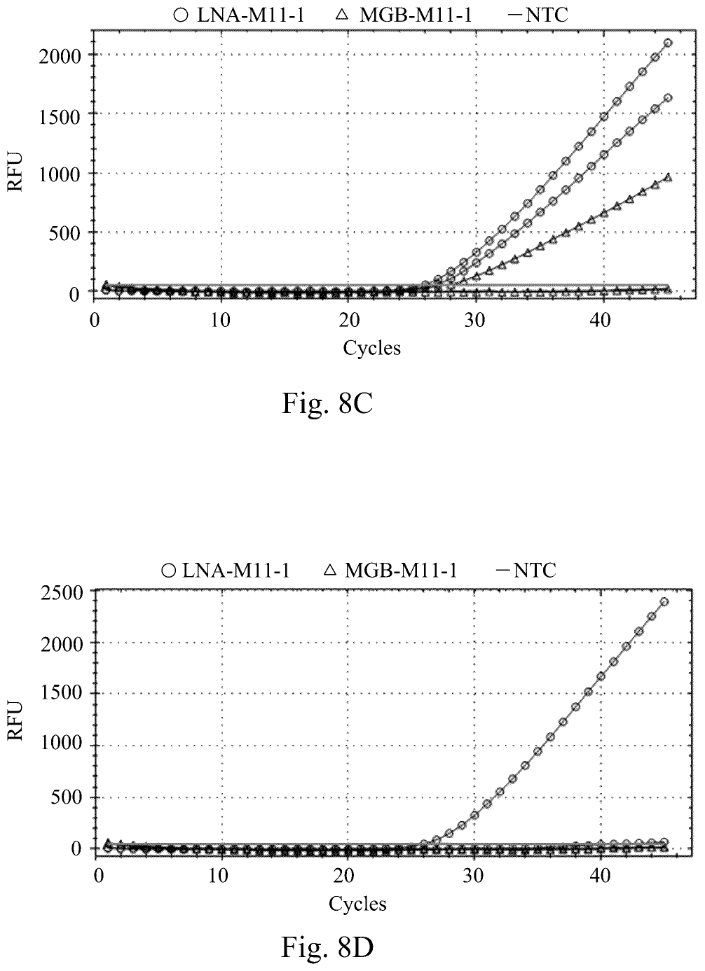

The reaction solutions were prepared according to Table 12, and 100 copies, 10 copies, 5 copies, and 2 copies of S531W mutation template plasmid were added, respectively. The amplification reactions were performed according to the PCR reaction procedure in Table 7, using Biorad CFX96 Real-time PCR Detection system. The results are shown in FIG. 8A (100 copies of plasmid), FIG. 8B (10 copies of plasmid), FIG. 8C (5 copies of plasmid), and FIG. 8D (2 copies of plasmid).

TABLE 23
Multiplex PCR mode for detecting
S531L, S531W, and L533P mutations

| Component | Volume (μl) | Final concentration | |
|---|---|---|---|
| QuantiNova reaction mixture | 13.5 | | |
| Primer Nest PF (50 μM) | 0.3 | 600 | nM |
| Primer Nest PR (50 μM) | 0.3 | 600 | nM |
| Primer RpoB PF (50 μM) | 0.3 | 600 | nM |
| Primer RpoB PR (50 μM) | 0.3 | 600 | nM |
| MGB-M10-1 (10 μM) | 1 | 400 | nM |
| MGB-M11-1 or | 1 | 400 | nM |
| LNA-M11-1C (10 μM) | | | |
| MGB-M12-1 (10 μM) | 0.5 | 200 | nM |
| PNAs 531 and 533 (10 μM) | 1 | 400 | nM |
| Internal control primer PF (10 μM) | 0.375 | 150 | nM |
| Internal control primer PR (10 μM) | 0.375 | 150 | nM |
| Internal control probe (10 μM) | 0.1875 | 75 | nM |
| Internal control template | 0.15 | 75 | copies |

| | | |
|---|---|---|
| DNA (500 copies/μl) | | |
| S531W mutation plasmid | 5 | |
| dH₂O | 0.7125 | |
| Total volume | 25 | — |

PNAs 531 and 533 are blocker probes used to block the amplification of the wild-type template at codons 531 and 533.

The amplification Cq values and RFU values are shown in Table 24. In all testing conditions with different copy numbers of the mutation template plasmids added, the Cq values and RFU values of the LNA probe LNA-M11-1C were significantly better than those of the MGB probe MGB-M11-1. In addition, in the case of multiplex reactions, the lower limit concentration of the detected mutant template for LNA probe LNA-M11-1C remains relatively low. When 5 copies of the mutant template plasmid were added, in the reaction using probe MGB-M11-1, the amplification was unstable; in contrast, in the reaction using LNA probe LNA-M11-1C, the mutant template was still stably amplified. When the number of copies of mutant template plasmid decreased to 2 copies, in the reaction using probe MGB-M11-1, the mutant template no longer be amplified; whereas there was still an amplification signal in the reaction using LNA probe LNA-M11-1C, indicating that the mutant template was amplified. This indicates that the optimized mutation detection LNA probes can effectively improve the detection sensitivity and better meet clinical detection requirements.

TABLE 24
Sensitivity test of LNA probe amplification
for S531W mutation plasmid

| S531W | MGB-M11-1 probe | | | LNA-M11-1C Probe | | |
|---|---|---|---|---|---|---|
| plasmid | FAM-Cq | Average | | FAM-Cq | Average | |
| copies | value | Cq value | RFU | value | Cq value | RFU |
| 100 | 22.64 | 23.63 | 1000 | 22.08 | 22.06 | 2800 |
| | 24.61 | | | 22.04 | | |
| 10 | 25.9 | 26.50 | 1000 | 24.67 | 24.62 | 2500 |
| | 27.09 | | | 24.57 | | |
| 5 | N/A | 27.42 | 1000 | 26.54 | 26.11 | 1700 |
| | 27.42 | | | 25.68 | | |
| 2 | N/A | — | — | 25.87 | 25.87 | 2500 |
| | N/A | | | N/A | | |
| NTC | NA | — | — | NA | — | — |

### 3.2 Testing of LNA Probes in Combination with MGB Probes

In this example, five types of mutations, including L511P mutation, Q513P mutation, H526D mutation, H526L mutation and H526R mutation, were detected simultaneously by adding five detection probes into one tube. To compare the amplification effects of LNA probes and MGB probes in combination, two probe groups were tested: the MGB probe group and the LNA probe group. Herein, the LNA probe group refers to a group of probes in which at least one of the probes is an LNA probe. The MGB probe group includes five MGB probes (MGB-M1-1, MGB-M3-1, MGB-M6-1, MGB-M7-1, and MGB-M8-1). The LNA probe group includes three MGB probes (MGB-M1-1, MGB-M3-1, and MGB-M8-1) and two LNA probes (LNA-M6-1B and LNA-M7-1). The probe sequences are shown in Table 25. The bold text shows the codon sequence corresponding to the mutation site, the bold italic text corresponds to the specific mutation site, and [N] indicates the site modified with LNA.

TABLE 25

| Probe | Mutation type | Probe Sequence (5′ > 3′) | SEQ ID number |
|---|---|---|---|
| MGB-M1-1 | L511P | CAGCCGAGCCAAT | SEQ ID NO: 1 |
| MGB-M3-1 | Q513P | CTGAGCCCATTCAT | SEQ ID NO: 5 |
| MGB-M6-1 | H526D | CGCTTGTCGGTCAA | SEQ ID NO: 11 |
| MGB-M7-1 | H526L | CTTGAGGGTCAACC | SEQ ID NO: 13 |
| MGB-M8-1 | H526R | CTTGCGGGTCAAC | SEQ ID NO: 15 |
| LNA-M6-1B | H526D | CG[C]T[T]G[TC]G[G]TCAA | SEQ ID NO: 40 |
| LNA-M7-1 | H526L | C[T]T[GA]G[G]G[T]C[A]ACC | SEQ ID NO: 45 |

The reaction solutions were prepared according to Table 26. In different tubes, L511P mutation template plasmid, Q513P mutation template plasmid, H526D mutation template plasmid, H526L mutation template plasmid, and H526R mutation template plasmid were added respectively. The amplification reactions were performed according to the PCR reaction procedure in Table 7, using Biorad CFX96 Real-time PCR Detection system.

TABLE 26
Multiplex PCR mode for detecting L511P,
Q513P, H526D, H526L, and H526R mutations

| Component | Volume (μl) | Final concentration | |
|---|---|---|---|
| QuantiNova reaction mixture | 13.5 | | |
| Primer Nest PF (50 μM) | 0.3 | 600 | nM |
| Primer Nest PR (50 μM) | 0.3 | 600 | nM |
| Primer RpoB PF (50 μM) | 0.3 | 600 | nM |
| Primer RpoB PR (50 μM) | 0.3 | 600 | nM |
| MGB-M1-1 (10 μM) | 0.5 | 200 | nM |
| MGB-M3-1 (10 μM) | 0.5 | 200 | nM |
| MGB-M6-1 or LNA-M6-1B (2 μM) | 2.5 | 200 | nM |
| MGB-M7-1 or LNA-M7-1 (2 μM) | 2.5 | 200 | nM |
| MGB-M8-1 (10 μM) | 0.5 | 200 | nM |
| PNAs 511 + 513(50 μM) | 0.2 | 400 | nM |
| PNA526 (10 μM) | 1 | 400 | nM |
| Internal control primer PF (10 μM) | 0.375 | 150 | nM |
| Internal control primer PR (10 μM) | 0.375 | 150 | nM |
| Internal control probe (10 μM) | 0.1875 | 75 | nM |
| Internal control template DNA (500 | 0.15 | 75 | copies |

| | | |
|---|---|---|
| copies/μl) | | |
| Mutation template plasmid | 1 | |
| ddH2O | 0.5125 | |
| Total volume | 25 | — |

PNAs 511 and 513 are blocker probes used to block amplification of the wild-type template at codons 511 and 513.

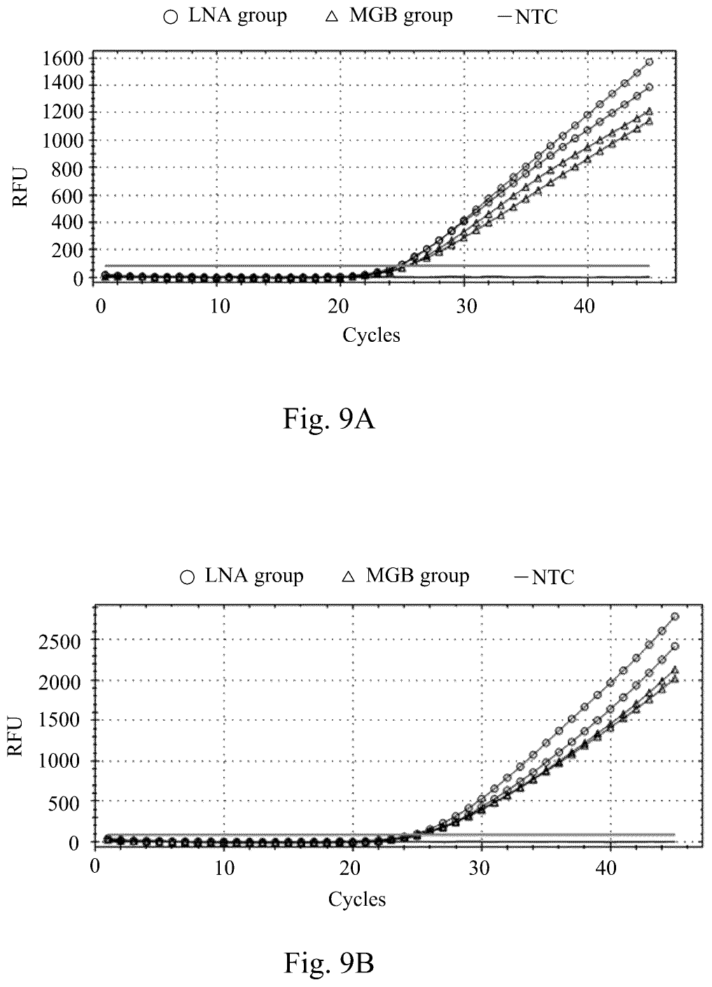

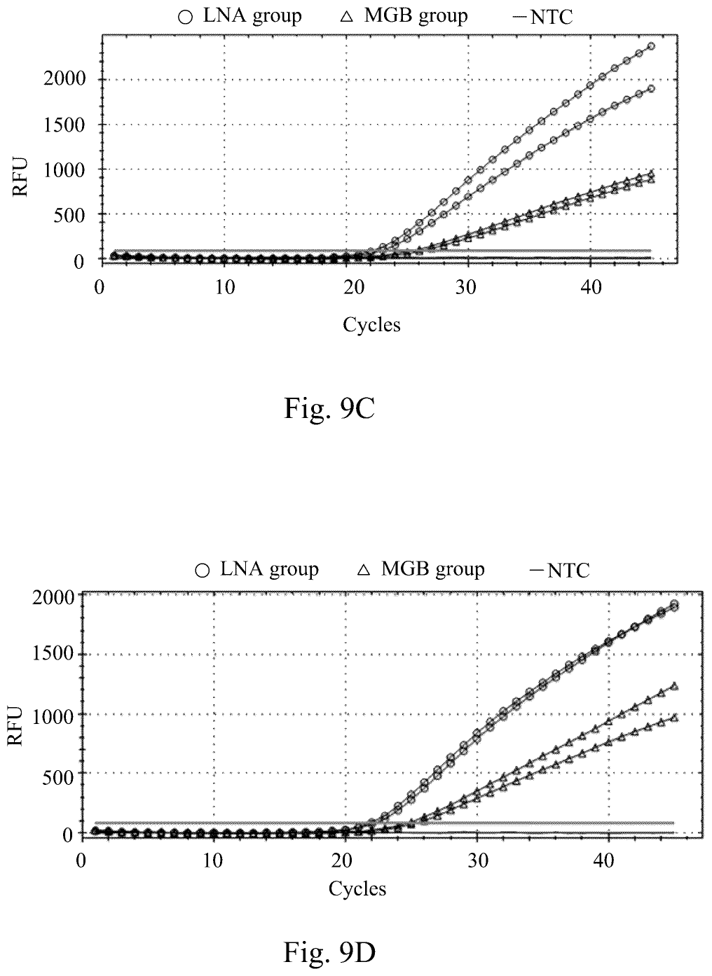

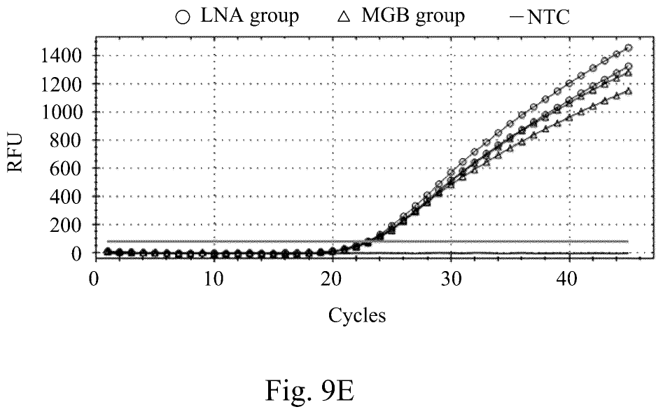

The amplification results are shown in FIG. 9A (L511P mutation), FIG. 9B (Q513P mutation), FIG. 9C (H526D mutation), FIG. 9D (H526L mutation), and FIG. 9E (H526R mutation). The Cq values and RFU values are summarized in Table 27. In this co-tube multiplex detection, the use of probe LNA-M6-1B and probe LNA-M7-1 significantly improved the amplification efficiency for H526D and H526L mutation plasmids, with earlier Cq values and significantly higher RFU. Additionally, the amplification Cq values for L511P mutation, Q513P mutation, and H526R mutation, which were detected using MGB probes, also showed some improvement.

TABLE 27
Results of multiplex PCR mode for detecting L511P,
Q513P, H526D, H526L, and H526R mutations

| Mutation | MGB Probe Group | | | LNA Probe Group | | |
|---|---|---|---|---|---|---|
| template | FAM-Cq | Average | | FAM | Average | |
| plasmid | value | Cq value | RFU | value | Cq value | RFU |
| L511P | 25.48 | 25.49 | 1200 | 24.73 | 24.79 | 1400 |
| | 25.49 | | | 24.85 | | |
| Q513P | 24.91 | 25.05 | 2000 | 25.29 | 24.94 | 2500 |
| | 25.18 | | | 24.59 | | |
| H526D | 26.27 | 25.90 | 1000 | 23.01 | 22.60 | 2000 |
| | 25.52 | | | 22.19 | | |
| H526L | 25.39 | 25.13 | 1000 | 21.91 | 22.12 | 2000 |
| | 24.86 | | | 22.33 | | |
| H526R | 23.11 | 23.20 | 1200 | 23.24 | 23.10 | 1300 |
| | 23.28 | | | 22.95 | | |

The above results indicate that the optimized LNA probes, compared to MGB probes, not only improve the detection sensitivity for the corresponding mutation types but also do not significantly inhibit the amplification of other mutation types in the co-tube multiplex detection. In reagent development, multiple probes respectively targeting different mutation types can be flexibly combined to achieve multiplex detection, reducing the difficulty of reagent development.

In some embodiments, different probe combinations can be tested for multiplex detection. Since LNA-modified probes are more expensive, in multiplex detection, LNA-modified probes and MGB probes can be combined to meet the sensitivity requirements of the detection.

In some embodiments, when multiplex detection is conducted, multiple probes respectively targeting closely located sites in the same PCR tube may exhibit suppression effects. Therefore, these probes that may cause suppression effects are placed in different PCR tubes (i.e., in separate PCR solutions). In some embodiments, the second probe M2-1 is not in the same tube as the first probe M1-1 and the third probe M3-1. In some embodiments, the ninth probe M9-1 is not be in the same tube as the sixth probe M6-1, seventh probe M7-1, and eighth probe M8-1.

In some embodiments, 2 to 6 probes are added to at least one reaction tube. In some embodiments, at least one probe in the same tube is an LNA probe. Other probes in the same tube may be MGB probes.

In some embodiments, 12 probes are used to detect the 12 mutation sites at 6 codons, such as the first probe (M1-1, M1-2), second probe (M2-1, M2-2) . . . twelfth probe (M12-1, M12-2) shown in Table 2. These 12 probes can be added individually to separate reaction tubes for individual reactions, i.e., one probe in one PCR tube. Alternatively, multiple probes, such as 2 to 6 probes, can be added into a single reaction tube.

In some embodiments, the twelve probes shown in Table 2 are divided into two groups, and the mutation detection is performed in two PCR tubes or two PCR solutions. Additionally, one or more of these probes are modified with LNA. In the first reaction tube, the second probe, fourth probe, fifth probe, ninth probe, tenth probe, and twelfth probe are added; in the second reaction tube, the first probe, third probe, sixth probe, seventh probe, eighth probe, and eleventh probe are added.

In some embodiments, the twelve probes shown in Table 2 are divided into three groups, and the mutation detection is performed in three PCR tubes or three PCR solutions. Additionally, one or more of these probes are modified with LNA. In the first reaction tube, the second probe, fourth probe, fifth probe, and ninth probe are added; in the second reaction tube, the first probe, third probe, sixth probe, seventh probe, and eighth probe are added; in the third reaction tube, the tenth probe, eleventh probe, and twelfth probe are added.

The probe combinations and detection methods provided in the embodiments of the present disclosure have several advantages, such as directly determining the presence of drug resistance mutations in the sample based on the amplification signal, identifying the specific drug resistance mutation type, and simultaneously detecting multiple drug resistance mutations (i.e., more than one mutation site) in a single reaction tube.

Although the present invention has been described in considerable detail with reference to certain embodiments thereof, other embodiments are possible.

Therefore, the spirit and scope of the appended claims should not be limited to the description of the embodiments contained herein. It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

What is claimed is:

1. A nucleic acid probe combination for detecting rifampicin resistance gene mutation in Mycobacterium tuberculosis, comprising:
a plurality of probes for a nucleic acid amplification reaction, wherein each of the plurality of probes is completely complementary to one of mutant sequence segments in a drug resistance-determining region of rpoB gene, and at least one of the plurality of probes has an LNA modification.

2. The nucleic acid probe combination for detecting rifampicin resistance gene mutation in Mycobacterium tuberculosis of claim 1, wherein each of the plurality of probes is configured to detect a mutation at one of codon 511, codon 513, codon 516, codon 526, codon 531, or codon 533 of the rpoB gene.

3. The nucleic acid probe combination for detecting rifampicin resistance gene mutation in Mycobacterium tuberculosis of claim 1, wherein each of the plurality of probes corresponds to one of L511P mutation, Q513K mutation, Q513P mutation, D516V mutation, D516Y mutation, H526D mutation, H526L mutation, H526R mutation, H526Y mutation, S531L mutation, S531W mutation, and L533P mutation.

4. The nucleic acid probe combination for detecting rifampicin resistance gene mutation in Mycobacterium tuberculosis of claim 1, wherein the LNA modification includes 3 to 8 LNA-modified bases located on both sides of a mutated base.

5. The nucleic acid probe combination for detecting rifampicin resistance gene mutation in Mycobacterium tuberculosis of claim 1, wherein the LNA modification includes 3 LNA-modified nucleotides within a range of 2 bases on both sides of a mutation base.

6. The nucleic acid probe combination for detecting rifampicin resistance gene mutation in Mycobacterium tuberculosis of claim 1, wherein another one of the plurality of probes has an MGB modification or is a Taqman probe.

7. The nucleic acid probe combination for detecting rifampicin resistance gene mutation in Mycobacterium tuberculosis of claim 1, wherein each of the plurality of probes comprises:
a fluorescent group at a 5′ end; and
a quencher group at a 3′ end.

8. The nucleic acid probe combination for detecting rifampicin resistance gene mutation in Mycobacterium tuberculosis of claim 1, wherein the plurality of probes comprise:
a first probe including a sequence of SEQ ID NO: 1 or 2;
a second probe including a sequence of SEQ ID NO: 3 or 4;
a third probe including a sequence of SEQ ID NO: 5 or 6;
a fourth probe including a sequence of SEQ ID NO: 7 or 8;
a fifth probe including a sequence of SEQ ID NO: 9 or 10;
a sixth probe including a sequence of SEQ ID NO: 11 or 12;
a seventh probe including a sequence of SEQ ID NO: 13 or 14;
an eighth probe including a sequence of SEQ ID NO: 15 or 16;
a ninth probe including a sequence of SEQ ID NO: 17 or 18;
a tenth probe including a sequence of SEQ ID NO: 19 or 20;
an eleventh probe including a sequence of SEQ ID NO: 21 or 22; or
a twelfth probe including a sequence of SEQ ID NO: 23 or 24.

9. The nucleic acid probe combination for detecting rifampicin resistance gene mutation in Mycobacterium tuberculosis of claim 8, wherein each of the first probe, the second probe, the third probe, the fourth probe, the fifth probe, the sixth probe, the seventh probe, the eighth probe, the ninth probe, the tenth probe, the eleventh probe, and the twelfth probe has LNA-modified bases.

10. The nucleic acid probe combination for detecting rifampicin resistance gene mutation in Mycobacterium tuberculosis of claim 9, wherein the first probe has a sequence of SEQ ID NO: 25 or 26; the second probe has a sequence of SEQ ID NO: 27 or 28; the third probe has a sequence of SEQ ID NO: 29 or 30; the fourth probe has a sequence of one of SEQ ID NO: 31 to 36; the fifth probe has a sequence of SEQ ID NO: 37 or 38; the sixth probe has a sequence of one of SEQ ID NO: 39 to 44; the seventh probe has a sequence of SEQ ID NO: 45 or 46; the eighth probe has a sequence of SEQ ID NO: 47 or 48; the ninth probe has a sequence of SEQ ID NO: 49 or 50; the tenth probe has a sequence of SEQ ID NO:
51 or 52; the eleventh probe has a sequence of one of SEQ ID NO: 53 to 60; and
the twelfth probe has a sequence of SEQ ID NO: 61 or 62.

11. A method for detecting rifampicin resistance gene mutation in Mycobacterium tuberculosis, comprising:
obtaining a DNA sample to be tested;
performing a nucleic acid amplification reaction, comprising: performing the nucleic acid amplification reaction using a nucleic acid probe combination, wherein the nucleic acid probe combination includes a plurality of probes, wherein each of the plurality of probes is completely complementary to one of mutant sequence segments in a drug resistance-determining region of rpoB gene, and at least one of the plurality of probes has an LNA modification.

12. The method for detecting rifampicin resistance gene mutation in Mycobacterium tuberculosis of claim 11, wherein each of the plurality of probes corresponds to one of L511P mutation, Q513K mutation, Q513P mutation, D516V mutation, D516Y mutation, H526D mutation, H526L mutation, H526R mutation, H526Y mutation, S531L mutation, S531W mutation, and L533P mutation.

13. The method for detecting rifampicin resistance gene mutation in Mycobacterium tuberculosis of claim 11, wherein the plurality of probes comprise:
a first probe including a sequence of SEQ ID NO: 1 or 2;
a second probe including a sequence of SEQ ID NO: 3 or 4;
a third probe including a sequence of SEQ ID NO: 5 or 6;
a fourth probe including a sequence of SEQ ID NO: 7 or 8;
a fifth probe including a sequence of SEQ ID NO: 9 or 10;
a sixth probe including a sequence of SEQ ID NO: 11 or 12;
a seventh probe including a sequence of SEQ ID NO: 13 or 14;
an eighth probe including a sequence of SEQ ID NO: 15 or 16;
a ninth probe including a sequence of SEQ ID NO: 17 or 18;
a tenth probe including a sequence of SEQ ID NO: 19 or 20;
an eleventh probe including a sequence of SEQ ID NO: 21 or 22; or
a twelfth probe including a sequence of SEQ ID NO: 23 or 24.

14. The method for detecting rifampicin resistance gene mutation in Mycobacterium tuberculosis of claim 13, wherein each of the first probe, the second probe, the third probe, the fourth probe, the fifth probe, the sixth probe, the seventh probe, the eighth probe, the ninth probe, the tenth probe, the eleventh probe, and the twelfth probe has LNA-modified bases.

15. The method for detecting rifampicin resistance gene mutation in Mycobacterium tuberculosis of claim 13, wherein the first probe has a sequence of SEQ ID NO: 25 or 26; the second probe has a sequence of SEQ ID NO: 27 or 28; the third probe has a sequence of SEQ ID NO: 29 or 30; the fourth probe has a sequence of one of SEQ ID NO: 31 to 36; the fifth probe has a sequence of SEQ ID NO: 37 or 38; the sixth probe has a sequence of one of SEQ ID NO: 39 to 44; the seventh probe has a sequence of SEQ ID NO: 45 or 46; the eighth probe has a sequence of SEQ ID NO: 47 or 48; the ninth probe has a sequence of SEQ ID NO: 49 or 50; the tenth probe has a sequence of SEQ ID NO: 51 or 52; the eleventh probe has a sequence of one of SEQ ID NO: 53 to 60; and the twelfth probe has a sequence of SEQ ID NO: 61 or 62.

16. The method for detecting rifampicin resistance gene mutation in Mycobacterium tuberculosis of claim 13, wherein the nucleic acid amplification reaction is performed using the first to the twelfth probes, and a reaction solution is prepared in at least two tubes, each of the at least two tubes containing one to six of the first to the twelfth probes.

17. The method for detecting rifampicin resistance gene mutation in Mycobacterium tuberculosis of claim 13, wherein at least one of the plurality of probes in a same tube has the LNA modification.

18. The method for detecting rifampicin resistance gene mutation in Mycobacterium tuberculosis of claim 13, wherein the second probe is not in a same tube as the first probe and the third probe.

19. The method for detecting rifampicin resistance gene mutation in Mycobacterium tuberculosis of claim 13, wherein the ninth probe is not in a same tube as the sixth probe, seventh probe, and eighth probe.

20. The method for detecting rifampicin resistance gene mutation in Mycobacterium tuberculosis of claim 13, wherein:
adding the second probe, fourth probe, fifth probe, ninth probe, tenth probe, and twelfth probe in a first reaction tube; and
adding the first probe, third probe, sixth probe, seventh probe, eighth probe, and eleventh probe to a second reaction tube.

21. The method for detecting rifampicin resistance gene mutation in Mycobacterium tuberculosis of claim 13, wherein:
adding the second probe, fourth probe, fifth probe, and ninth probe to a first reaction tube;
adding the first probe, third probe, sixth probe, seventh probe, and eighth probe to a second reaction tube; and
adding the tenth probe, eleventh probe, and twelfth probe to a third reaction tube.
